SEQUENCE LISTING (4 sequences)

SEQ ID NO: 1 - DNA, 682 bp, human
  CDS (1)..(678)
atggtcacccacagcaagtttcccgccgccgggatgagccgccccctggacaccagcctgcgcctcaagaccttcagctccaagagcgagtaccagctggtggtgaacgcagtgcgcaagctgcaggagagcggcttctactggagcgcagtgaccggcggcgaggcgaacctgctgctcagcgccgagcccgccggcacctttctgatccgcgacagctcggaccagcgccacttcttcacgctcagcgtcaagacccagtctgggaccaagaacctgcgcatccagtgtgaggggggcagcttctctctgcagagcgatccccggagcacgcagcccgtgccccgcttcgactgcgtgctcaagctggtgcaccactacatgccgccccctggagccccctccttcccctcgccacctactgaaccctcctccgaggtgcccgagcagccgtctgcccagccactccctgggagtccccccagaagagcctattacatctactccgggggcgagaagatccccctggtgttgagccggcccctctcctccaacgtggccactcttcagcatctctgtcggaagaccgtcaacggccacctggactcctatgagaaagtcacccagctgccggggcccattcgggagttcctggaccagtacgatgccccgctttaagggg

SEQ ID NO: 2 - protein, 225 aa, human
MVTHSKFPAAGMSRPLDTSLRLKTFSSKSEYQLVVNAVRKLQESGFYWSAVTGGEANLLLSAEPAGTFLIRDSSDQRHFFTLSVKTQSGTKNLRIQCEGGSFSLQSDPRSTQPVPRFDCVLKLVHHYMPPPGAPSFPSPPTEPSSEVPEQPSAQPLPGSPPRRAYYIYSGGEKIPLVLSRPLSSNVATLQHLCRKTVNGHLDSYEKVTQLPGPIREFLDQYDAPL

SEQ ID NO: 3 - DNA, 2187 bp, murine
  CDS (18)..(692)
cgctggctccgtgcgccatggtcacccacagcaagtttcccgccgccgggatgagccgccccctggacaccagcctgcgcctcaagaccttcagctccaaaagcgagtaccagctggtggtgaacgccgtgcgcaagctgcaggagagcggattctactggagcgccgtgaccggcggcgaggcgaacctgctgctcagcgccgagcccgcgggcacctttcttatccgcgacagctcggaccagcgccacttcttcacgttgagcgtcaagacccagtcggggaccaagaacctacgcatccagtgtgaggggggcagcttttcgctgcagagtgacccccgaagcacgcagccagttccccgcttcgactgtgtactcaagctggtgcaccactacatgccgcctccagggaccccctccttttctttgccacccacggaaccctcgtccgaagttccggagcagccacctgcccaggcactccccgggagtacccccaagagagcttactacatctattctgggggcgagaagattccgctggtactgagccgacctctctcctccaacgtggccaccctccagcatctttgtcggaagactgtcaacggccacctggactcctatgagaaagtgacccagctgcctggacccattcgggagttcctggatcagtatgatgctccactttaaggagcaaaagggtcagaggggggcctgggtcggtcggtcgcctctcctccgaggcacatggcacaagcacaaaaatccagccccaacggtcggtagctcccagtgagccaggggcagattggcttcttcctcaggccctccactcccgcagagtagagctggcaggacctggaattcgtctgaggggagggggagctgccacctgctttcccccctcccccagctccagcttctttcaagtggagccagccggcctggcctggtgggacaatacctttgacaagcggactctcccctccccttcctccacaccccctctgcttcccaagggaggtggggacacctccaagtgttgaacttagaactgcaaggggaatcttcaaactttcccgctggaacttgtttgcgctttgatttggtttgatcaagagcaggcacctgggggaaggatggaagagaaaagggtgtgtgaagggtttttatgctggccaaagaaataaccactcccactgcccaacctaggtgaggagtggtggctcctggctctggggagagtggcaaggggtgacctgaagagagctatactggtgccaggctcctctccatggggcagctaatgaaacctcgcagatcccttgcaccccagaaccctccccgttgtgaagaggcagtagcatttagaagggagacagatgaggctggtgagctggccgccttttccaacaccgaagggaggcagatcaacagatgagccatcttggagcccaggtttcccctggagcagatggagggttctgctttgtctctcctatgtggggctaggagactcgccttaaatgccctctgtcccagggatggggattggcacacaaggagccaaacacagccaataggcagagagttgagggattcacccaggtggctacaggccaggggaagtggctgcaggggagagacccagtcactccaggagactcctgagttaacactgggaagacattggccagtcctagtcatctctcggtcagtaggtccgagagcttccaggccctgcacagccctcctttctcacctggggggaggcaggaggtgatggagaagccttcccatgccgctcacaggggcctcacgggaatgcagcagccatgcaattacctggaactggtcctgtgttggggagaaacaagttttctgaagtcaggtatggggctgggtggggcagctgtgtgttggggtggcttttttctctctgttttgaataatgtttacaatttgcctcaatcacttttataaaaatccacctccagcccgcccctctccccactcaggccttcgaggctgtctgaagatgcttgaaaaactcaaccaaatcccagttcaactcagactttgcacatatatttatatttatactcagaaaagaaacatttcagtaatttataataaaagagcactattttttaatgaaaaaaaaaaaaaaaaaaaaaaaaaa

SEQ ID NO: 4 - protein, 225 aa, murine
MVTHSKFPAAGMSRPLDTSLRLKTFSSKSEYQLVVNAVRKLQESGFYWSAVTGGEANLLLSAEPAGTFLIRDSSDQRHFFTLSVKTQSGTKNLRIQCEGGSFSLQSDPRSTQPVPRFDCVLKLVHHYMPPPGTPSFSLPPTEPSSEVPEQPPAQALPGSTPKRAYYIYSGGEKIPLVLSRPLSSNVATLQHLCRKTVNGHLDSYEKVTQLPGPIREFLDQYDAPL

(19) United States
(12) Patent Application Publication
(10) Pub. No.: US 2007/0275916 A1
(43) Pub. Date: Nov. 29, 2007

(54) Methods of Identifying Compounds Which Modulate Granulocite-Colony Stimulating Factor (G-Csf) Dependent Processes by Modulation of the Levels of Suppressor of Cytokine Signaling (Socs)

(75) Inventors: Ben Croker, Tamworth (AU); Andrew Roberts, Camberwell (AU); Don Metcalf, Balwyn (AU); Warren Alexander, Moonee Ponds (AU); Doug Hilton, Warrandyte (AU); Nicos Nicola, Mont Albert (AU)

Correspondence Address: SCULLY, SCOTT, MURPHY & PRESSER, P.C., 400 GARDEN CITY PLAZA, SUITE 300, GARDEN CITY, NY, 11530, US

(73) Assignee: THE WALTER AND ELIZA HALL INSTITUTE OF MEDICAL RESEARCH

(21) Appl. No.: 10/559,711

(22) PCT Filed: Jun. 4, 2004
(86) PCT No.: PCT/AU04/00749

§ 371 Date: May 18, 2006

(30) Foreign Application Priority Data
Jun. 4, 2003 (AU) 2003902788

(51) Int. Cl. A61K 48/00; A01K 67/00; C07H 21/04; C07K 1/00

(52) U.S. Cl. 514/44; 530/350; 536/22.1; 536/23.1; 536/24.5; 800/9

(57) ABSTRACT

The present invention relates generally to compounds which modulate cytokine-dependent processes. More particularly, the compounds of the present invention modulate responses to a colony stimulating factor and even more particularly to granulocyte-colony stimulating factor (G-CSF) by modulating the levels of molecules which inhibit G-CSF such as but not limited to a suppressor of cytokine signaling (SOCS) and in particular SOCS-3. The present invention further contemplates methods for regulating G-CSF-dependent processes by contacting cells in vitro with or administering to a subject a compound which up- or down-regulates the level of activity of G-CSF by modulating the level or activity of a SOCS molecule such as SOCS-3. The instant compounds are further useful for modulating a range of G-CSF-induced cellular responses including neutrophil recovery after chemotherapy or radiotherapy, mobilizing stem and progenitor cells, treating infection and treating inflammatory conditions.

### BACKGROUND OF THE INVENTION

1. Field of the Invention

The present invention relates generally to compounds which modulate cytokine-dependent processes. More particularly, the compounds of the present invention modulate responses to a colony stimulating factor and even more particularly to granulocyte-colony stimulating factor (G-CSF) by modulating the levels of molecules which inhibit G-CSF such as but not limited to a suppressor of cytokine signaling (SOCS) and in particular SOCS-3. The present invention further contemplates methods for regulating G-CSF-dependent processes by contacting cells in vitro with or administering to a subject a compound which up- or down-regulates the level of activity of G-CSF by modulating the level or activity of a SOCS molecule such as SOCS-3. The instant compounds are further useful for modulating a range of G-CSF-induced cellular responses including neutrophil recovery after chemotherapy or radiotherapy, mobilizing stem and progenitor cells, treating infection and treating inflammatory conditions.

2. Description of the Prior Art

Bibliographic details of the publications referred to in this specification are also collected at the end of the description.

Reference to any prior art in this specification is not, and should not be taken as, an acknowledgment or any form of suggestion that this prior art forms part of the common general knowledge in any country.

The suppressor of cytokine signaling (SOCS) proteins are a family of eight SH2 domain containing proteins which includes the cytokine-inducible SH2 (CIS) domain-containing protein and SOCS-1 to 7. Studies in many laboratories have implicated SOCS proteins in the attenuation of cytokine action through inhibition of the Janus Kinase (JAK)/Signal Transducer and Activators of Transcription (STAT) signal transduction pathway. SOCS proteins operate as part of a classical negative feedback loop, in which activation of cytokine signaling leads to their expression. Once produced, SOCS proteins bind to key components of the signaling apparatus to deactivate and possibly target them for degradation via a conserved C-terminal motif, called the “SOCS Box”, that recruits ubiquitin ligases (reviewed in Krebs and Hilton, J. Cell Sci. 113(16): 2813-2819, 2000; Yasukawa et al., Annu. Rev. Immunol. 18: 143-164, 2000; Greenhalgh and Hilton, J. Leukoc. Biol. 70(3): 348-356, 2001).

While in vitro studies have suggested that SOCS proteins may be promiscuous in their activity, gene deletion studies in mice have highlighted their importance in a limited number of signaling pathways. SOCS-1 is a key regulator of IFN-γ signaling, T-cell homeostasis and lactation (Marine et al., Cell 98(5): 609-616, 1999; Alexander et al., Cell 98(5): 597-608, 1999; Lindeman et al., Genes Dev. 15(13): 1631-1636, 2001), while SOCS-3 is thought to play crucial roles in erythropoiesis and placental function (Marine et al., Cell 98(5): 617-627, 1999; Roberts et al., Proc. Natl. Acad. Sci. USA 98(16): 9324-9329, 2001). CIS-deficient mice are reported to have no phenotype, although CIS transgenic mice display growth retardation and defects in mammary development which are accompanied by reductions in STAT5 phosphorylation (Matsumoto et al., Mol. Cell Biol. 19(9): 6396-6407, 1999) and show similarities to the phenotypes observed in STAT5a and STAT5b deficient mice (Teglund et al., Cell 93(5): 841-850, 1998; Udy et al., Proc. Natl. Acad. Sci. USA 94(14): 7239-7244, 1997; Liu et al., Genes Dev. 11(2): 179-186, 1997).

SOCS-2 deficient animals exhibit accelerated post-natal growth resulting in a 30-50% increase in body weight by 12 weeks of age, significant increases in bone and body lengths, thickening of the skin due to collagen deposition and increases in internal organ size (Metcalf et al., Nature 405(6790): 1069-1073, 2000). This phenotype has striking similarities to those of insulin-like growth factor (IGF)-I and growth hormone (GH) transgenic mice (Palmiter et al., Science 222(4625): 809-814, 1983; Mathews et al., Endocrinology 123(6): 2827-2833, 1988). Further investigation of the SOCS-2^{-/-} phenotype identified significant increases of IGF-I mRNA in some tissues and lower levels of major urinary protein (MUP) the expression of which is regulated by intermittent GH secretion (Metcalf et al., 2000, supra). Recently, STAT5 phosphorylation in response to GH has been shown to be modestly prolonged in SOCS-2^{-/-} primary hepatocytes compared with to those from wild type mice, and much of the acceleration of growth in SOCS-2^{-/-} mice requires the presence of STAT5b, a key mediator of GH action (Greenhalgh et al., Molecular Endocrinology 16(6): 1394-1406, 2002).

All cellular responses to CSFs are the consequence of signals arising from the cytoplasmic domain of the CSF receptor (CSFR), after ligation of the extracellular domain of the receptor by a CSF. Granulocyte-colony stimulating factor (G-CSF) is an example of a CSF involved in a range of physiological processes such as inflammation and stem and progenitor cell mobilization. It is an essential regulator of normal neutrophil production and survival. Pharmacological therapy with recombinant human G-CSF is widely clinically used to accelerate neutrophil recovery after chemotherapy or hemopoietic stem cell transplantation, and to reduce the risk of development of life-threatening infections in these settings. G-CSF mediates its effects via its receptor, G-CSFR (Avalos, Blood 88(3): 761-777, 1996).

Ligation of the extracellular domain of the G-CSFR results in activation of multiple intracellular signaling cascades, some of which rely on phosphorylation of one or more of four tyrosine residues in the C-terminal region of the receptor. JAK1, JAK2 and TYK2 are tyrosine kinases recruited to the receptor, and these in turn activate STAT1, STAT3 and STAT5, among other signaling intermediates (Nicholson et al., Proc. Natl. Acad. Sci. USA 91(8): 2985-2988, 1994; Tian et al., Blood 84(6): 1760-1764, 1994; Tian et al., Blood 88(12): 4435-4444, 1996; de Koning et al., Blood 87(4): 1335-1342, 1996; Ward et al., Blood 93(1): 113-124, 1999).

Although it has been suspected that SOCS molecules and in particular SOCS-3 may have a role in regulating G-CSF, until the advent of the present invention, the importance of the SOCS molecule's involvement was unclear. For example, SOCS-3 expression is induced in primary myeloid cells when stimulated with G-CSF (Starr et al., Nature 387(6636): 917-921, 1997; Hortner et al., J. Immunol. 169(3): 1219-1927, 2002).

Furthermore, in cell-based over-expression systems, binding of SOCS-3 to G-CSFR inhibits STAT-dependent gene expression after stimulation of the cell with G-CSF (Hortner et al., 2002, supra). However, results derived from such over-expression systems are unreliable predictors of physiological processes. Examining cellular responses to G-CSF in the presence or absence of SOCS proteins such as SOCS-3 is a true test system enabling definition of the ability of modulators of SOCS-3 activity as regulators of G-CSF-induced responses.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. A summary of the sequence identifiers is provided in Table 1. A sequence listing is provided at the end of the specification.

Abbreviations used herein are defined in Table 2.

The present invention identifies a drug target useful in modulating CSF and in particular G-CSF-induced cellular processes. The instant invention is predicated in part on the identification of SOCS-3 as a key regulator of G-CSF intracellular signaling. The present invention enables, therefore, rational drug design or screening of natural product or chemical libraries for compounds which modulate the responses of cells to G-CSF. Enhancement of G-CSF-signaling is proposed to be useful in facilitating neutrophil recovery after myelosuppressive chemotherapy, radiotherapy or chemoradiotherapy, inducing mobilization of stem and progenitor cells and in the treatment of prophylaxis of bacterial, viral or fungal infection or recovery therefrom.

Down-regulation of G-CSF-signaling such as following administration of agonists of SOCS-3 is proposed to be useful in reducing unwanted clinical sequelae of inflammatory processes characterized by neutrophil accumulation and activation. Examples of such clinical sequelae include engraftment syndrome following allogeneic or autologous stem cell transplantation, pulmonary inflammation such as observed after recovery from neutropenia, acute arthritis, inflammatory bowel disease and Sweets syndrome.

The compounds of the present invention may be chemical molecules, peptides, polypeptides or proteins, or genetic molecules including nucleic acid molecules (such as sense and antisense molecules), RNAi or siRNA or complexes containing same. The compounds may also be formulated into a range of compositions.

The compounds of the present invention may be used to treat animals including mammalian animals such as human subjects with a range of G-CSF-mediated physiological conditions such as those listed above.

The present invention further provides methods for identifying compounds which up- or down-regulate G-CSF-signaling.

A summary of sequence identifiers used throughout the subject specification is provided in Table 1.

TABLE 1
Summary of sequence identifiers

| SEQUENCE ID NO: | DESCRIPTION |
|---|---|
| 1 | Nucleotide sequence encoding human SOCS-3 |
| 2 | Amino acid sequence of human SOCS-3 |
| 3 | Nucleotide sequence of mouse SOCS-3 |
| 4 | Amino acid sequence of mouse SOCS-3 |

TABLE 2
Abbreviations

| ABBREVIATION | DESCRIPTION |
|---|---|
| CSF | colony stimulating factor |
| G-CSF | granulocyte-colony stimulating factor |
| JAK | Janus kinase |
| SOCS | suppressor of cytokine signaling |
| SOCS Box | conserved C-terminal region of SOCS molecule |
| | which recruits ubiquitin ligases |
| STAT | signal transducer and activators of transcription |
| CIS | cytokine-inducible SH2 |
| IGF-I | insulin-like growth factor-I |
| GH | growth hormone |
| MUP | major urinary protein |
| CSFR | colony stimulating factor receptor |
| G-CSFR | granulocyte-stimulating factor receptor |
| bHLH | basic helix-loop-helix gene |
| BM | bone marrow |
| SOCS-3⁻ | null mutation in SOCS-3 allele |
| SOCS-3^{fl} | LoxP-flanked conditional SOCS-3 allele |

### BRIEF DESCRIPTION OF THE FIGURES

FIGS. 1 and 2 are photographic representations showing that hemopoietic cells from VavCre⁺ SOCS-3^{-/fl} mice are SOCS-3-deficient. DNA from bone marrow (BM), thymus, spleen and lymph nodes of VavCre⁺ SOCS^{+/fl}, VavCre⁺ SOCS-3^{-/fl} or VavCre⁻ SOCS-3^{+/fl} mice was extracted. In FIG. 1, Southern blotting reveals efficient deletion of the floxed (fl) allele with conversion to the Δ allele in all tissues examined. In FIG. 2, PCR or DNA from sub-fractionated BM cells was performed and the products revealed after electrophoresis in an ethidium-containing gel.

FIG. 3 is a graphical representation showing increased frequency of G-CSF-responsive CFC in SOCS-3-deficient bone marrow. 25,000 bone marrow cells, either SOCS-3 deficient or control were cultured with specific stimuli in supramaximal concentration for seven days. Mean ±SD of results from 4-6 mice per genotype. *p<0.01. Control mice had at least one functional SOCS-3 allele, i.e. genotype +/fl or +/Δ.

FIG. 4 is a graphical representation showing increased colony size in SOCS-3-deficient bone marrow stimulated by G-CSF and IL-6. 25,000 bone marrow cells, either SOCS-3-deficient or wild-type were cultured with specific stimuli in supramaximal concentrations for seven days, then individual colonies were picked, pooled and counted. Mean ±SD of results from 2-3 experiments. *p<0.01.

FIG. 5 is a graphical representation showing enhanced proliferation of SOCS-3-deficient Gr-1⁺ myeloid cells in response to G-CSF but not IL-3. 100,000 GR-1⁺ bone marrow cells, either SOCS-3-deficient or SOCS-3-sufficient were cultured with either G-CSF or IL-3 in various concentrations for 48 hours. Tritiated thymidine was then added and the cultures continued for a further 16 hours prior to analysis of thymidine incorporation. Mean ±SD of results of triplicate cultures per genotype.

FIG. 6 is a graphical representation showing enhanced in vivo responses induced by G-CSF in VavCre⁺ SOCS^{-/fl} mice. Mice were injected with G-CSF 2.5 μg twice daily i.p. for four days and analyzed on the fifth day. Progenitors were enumerated by culturing 5-20 μl of blood in standard CFC assays for seven days prior to fixation, staining and counting at 40× magnification. Mean ±SD of results from four mice per group (except vehicle injected VavCre⁺ SOCS^{-/fl} mice where n=2). *p<0.01.

### DETAILED DESCRIPTION OF THE INVENTION

The present invention provides compounds which selectively modulate levels or activity of a CSF and in particular G-CSF in animals, such as mammals and in particular humans. The compounds up-regulate or down-regulate intracellular signals induced by G-CSF. The compounds act by modulating levels or the activity of a SOCS molecule involved in inhibiting G-CSF-signaling, such as SOCS-3. Consequently, the present invention provides G-CSF modulators which include compounds which up- or down-regulate the levels or activity of SOCS-3 or other molecules affected by, or which affect, G-CSF activity. The G-CSF-signaling modulators of the present invention include agonists and antagonists of SOCS-3 and are useful in modulating G-CSF-induced physiological processes. For example, up-regulation of G-CSF-induced signaling is proposed to be useful in facilitating neutrophil recovery after myelosuppressive chemotherapy, radiotherapy or chemoradiotherapy, inducing mobilization of stem and progenitor cells and in the treatment of prophylaxis of bacterial, viral or fungal infection or recovery therefrom.

Down-regulation of G-CSF-induced signaling such as following administration of agonists of SOCS-3 is proposed to be useful in reducing unwanted clinical sequelae of inflammatory processes characterized by neutrophil accumulation and activation. Examples of such clinical sequelae include engraftment syndrome following allogenic or autologous stem cell transplantation, pulmonary inflammation such as observed after recovery from neutropenia, acute arthritis, inflammatory bowel disease and Sweets syndrome.

Before describing the present invention in detail, it is to be understood that unless otherwise indicated, the subject invention is not limited to specific formulation components, manufacturing methods, dosage regimens, or the like, as such may vary. It is also to be understood that the terminology used herein is for the purpose of describing particular embodiments only and is not intended to be limiting.

It must be noted that, as used in the subject specification, the singular forms “a”, “an” and “the” include plural aspects unless the context clearly dictates otherwise. Thus, for example, reference to a “compound” includes a single compound, as well as two or more compounds; reference to “an agonist” or “antagonist” includes a single agonist or antagonist as well as two or more agonists or antagonists, and so forth.

In describing and claiming the present invention, the following terminology will be used in accordance with the definitions set forth below.

The terms “compound”, “agonist”, “antagonist”, “active agent”, “pharmacologically active agent”, “medicament”, “active” and “drug” are used interchangeably herein to refer to a chemical compound that induces a desired pharmacological and/or physiological effect such a up- or down-regulating G-CSF-induced signaling or ameliorating the symptoms of elevated or reduced levels of G-CSF. The terms also encompass pharmaceutically acceptable and pharmacologically active ingredients of those active agents specifically mentioned herein including but not limited to salts, esters, amides, prodrugs, active metabolites, analogs and the like. When the terms “compound”, “agonist”, antagonist”, “active agent”, “pharmacologically active agent”, “medicament”, “active” and “drug” are used, then it is to be understood that this includes the active agent per se as well as pharmaceutically acceptable, pharmacologically active salts, esters, amides, prodrugs, metabolites, analogs, etc. The term “compound” is not to be construed as a chemical compound only but extends to peptides, polypeptides and proteins as well as genetic molecules such as RNA, DNA and chemical analogs thereof as well as RNAi- or siRNA-type molecules or complexes comprising same. Reference to “RNA” and “DNA” includes oligonucleotide RNA or DNA molecules as well as sense, antisense or double-stranded forms.

The present invention contemplates, therefore, compounds useful in up- or down-regulating G-CSF signaling via modulation of a SOCS molecule such as SOCS-3. One group of compounds acts as SOCS-3 antagonists which have the effect of up-regulating G-CSF signaling. Another group of compounds are SOCS-3 agonists which down-regulate G-CSF signaling. Yet another group of compounds affect gene expression of the SOCS-3 gene.

By the terms “effective amount” or “therapeutically effective amount” of an agent as used herein are meant a sufficient amount of the agent to provide the desired therapeutic or physiological effect. Furthermore, an “effective G-CSF modulating amount” of an agent is a sufficient amount of the agent to directly or indirectly reduce or elevate the levels of G-CSF-induced intracellular signaling. Indirect modulation in G-CSF induced intracellular signaling is conveniently achieved by up- or down-regulating SOCS-3 or providing a SOCS-3 equivalent or mimetic. Of course, undesirable effects, e.g. side effects, are sometimes manifested along with the desired therapeutic effect; hence, a practitioner balances the potential benefits against the potential risks in determining what is an appropriate “effective amount”. The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation.

By “pharmaceutically acceptable” carrier excipient or diluent is meant a pharmaceutical vehicle comprised of a material that is not biologically or otherwise undesirable, i.e. the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like.

Similarly, a “pharmacologically acceptable” salt, ester, amide, prodrug or derivative of a compound as provided herein is a salt, ester, amide, prodrug or derivative that is not biologically or otherwise undesirable.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual by inhibiting or causing regression of a disorder or disease. Thus, for example, the present method of “treating” a patient in need of therapy of conditions involving G-CSF-induced physiological processes encompasses both prevention of a condition, disease or disorder as well as treating the condition, disease or disorder. In any event, the present invention contemplates the treatment or prophylaxis of any condition requiring the up- or down-regulation of G-CSF-induced intracellular signaling and hence activity. Up-regulation of G-CSF-induced responses is proposed to be useful in facilitating neutrophil recovery after myelosuppressive chemotherapy, radiotherapy or chemoradiotherapy, inducing mobilization of stem and progenitor cells and in the treatment of prophylaxis of bacterial, viral or fungal infection or recovery therefrom.

Down-regulation of G-CSF-induced responses such as following administration of agonists of SOCS-3 is proposed to be useful in reducing unwanted clinical sequelae of inflammatory processes characterized by neutrophil accumulation and activation. Examples of such clinical sequelae include engraftment syndrome following allogenic or autologous stem cell transplantation, pulmonary inflammation such as observed after recovery from neutropenia, acute arthritis, inflammatory bowel disease and Sweets syndrome.

The present invention contemplates autologous therapy involving the removal of stem or progenitor cells, subjecting same to proliferation conditions, genetic manipulation or other physiological stimulus and then returning the cells to the same or a compatible subject in the presence of a compound of the present invention which facilitates mobilization of stem or progenitor cells. Autologous therapy is a form of treating a patient or subject.

“Patient” as used herein refers to an animal, preferably a mammal preferably a higher or lower primate and most preferably a human who can benefit from the pharmaceutical formulations and methods of the present invention. There is no limitation on the type of animal that could benefit from the presently described pharmaceutical formulations and methods. A patient regardless of whether a human or non-human animal may be referred to as an individual, subject, animal, host or recipient.

The compounds of this aspect of the present invention may be large or small molecules, nucleic acid molecules (including antisense or sense molecules) such as mRNA, cDNA, siRNA or RNAi, peptides, polypeptides or proteins or hybrid molecules such as RNAi- or siRNA-complexes, ribozymes or DNAzymes.

The preferred animals are humans or other primates, lower primates, livestock animals, laboratory test animals, companion animals or captive wild animals.

Examples of non-human primates include baboons and marmosets.

Examples of laboratory test animals include mice, rats, rabbits, guinea pigs and hamsters. Rabbits and rodent animals, such as rats and mice, provide a convenient test system or animal model as do primates and lower primates. Livestock animals include sheep, cows, pigs, goats, horses and donkeys. Non-mammalian animals such as zebrafish and amphibians (including cane toads) are also contemplated.

The present invention provides, therefore, drugs which inhibit or promote G-CSF activity by up- or down-regulating SOCS-3 activity or SOCS-3 gene expression.

The present invention contemplates, therefore, methods of screening for drugs comprising, for example, contacting a candidate drug with a G-CSF regulator molecule (e.g. SOCS-3) or a fragment thereof or a nucleic acid molecule encoding same. These molecules are referred to herein as “targets”, “a target” or “target molecule”. The screening procedure includes assaying (i) for the presence of a complex between the drug and the target, or (ii) an alteration in the expression levels of nucleic acid molecules encoding the target. One form of assay involves competitive binding assays. In such competitive binding assays, the target is typically labeled. Free target is separated from any putative complex and the amount of free (i.e. uncomplexed) label is a measure of the binding of the agent being tested to target molecule. One may also measure the amount of bound, rather than free, target. It is also possible to label the compound rather than the target and to measure the amount of compound binding to target in the presence and in the absence of the drug being tested.

Another technique for drug screening provides high throughput screening for compounds having suitable binding affinity to a target and is described in detail in Geysen (International Patent Publication No. WO 84/03564). Briefly stated, large numbers of different small peptide test compounds are synthesized on a solid substrate, such as plastic pins or some other surface. The peptide test compounds are reacted with a target and washed. Bound target molecule is then detected by methods well known in the art. This method may be adapted for screening for non-peptide, chemical entities. This aspect, therefore, extends to combinatorial approaches to screening for target antagonists or agonists.

Purified target can be coated directly onto plates for use in the aforementioned drug screening techniques. However, non-neutralizing antibodies to the target may also be used to immobilize the target on the solid phase.

The present invention also contemplates the use of competitive drug screening assays in which neutralizing antibodies capable of specifically binding the target compete with a test compound for binding to the target or fragments thereof. In this manner, the antibodies can be used to detect the presence of any peptide which shares one or more antigenic determinants of the target.

The above screening methods are particularly useful for screening for agents which interact with SOCS-3 and up-or-down-regulate activity or gene expression.

The present invention contemplates, therefore, any compound which inhibits G-CSF signaling within cells and which, therefore, modulates cellular responses to G-CSF.

Accordingly, one aspect of the present invention provides an isolated compound which inhibits or elevates G-CSF-induced responses.

As stated above, the present invention is also useful for screening for other compounds which up-regulates expression of a gene encoding SOCS-3, or which mimic SOCS-3 activity. Such targets may be used in any of a variety of drug screening techniques, such as those described herein and in International Publication No. WO 97/02048.

A target antagonist or agonist includes a variant of the target molecule. In one embodiment, the target is a polypeptide. The term “polypeptide” refers to a polymer of amino acids and its equivalent and does not refer to a specific length of the product, thus, peptides, oligopeptides and proteins are included within the definition of a polypeptide. This term also does not exclude modifications of the polypeptide, for example, glycosylations, acylations, phosphorylations and the like. Included within the definition are, for example, polypeptides containing one or more analogs of an amino acid (including, for example, unnatural amino acids, etc.), polypeptides with substituted linkages as well as other modifications known in the art, both naturally and non-naturally occurring. Ordinarily, such polypeptides will be at least about 40% similar to the natural target sequence, preferably in excess of 90% and more preferably at least about 95% similar. Also included are proteins encoding by DNAs which hybridize under high or low stringency conditions to target-encoding nucleic acids and closely related polypeptides or proteins retrieved by antisera to a target molecule protein.

Substitutional variants typically contain the exchange of one amino acid for another at one or more sites within the protein and may be designed to modulate one or more properties of the polypeptide such as stability against proteolytic cleavage without the loss of other functions or properties. Amino acid substitutions may be made on the basis of similarity in polarity, charge, solubility, hydrophobicity, hydrophilicity and/or the amphipathic nature of the residues involved. Preferred substitutions are ones which are conservative, that is, one amino acid is replaced with one of similar shape and charge. Conservative substitutions are well known in the art and typically include substitutions within the following groups: glycine, alanine; valine, isoleucine, leucine; aspartic acid, glutamic acid; asparagine, glutamine; serine, threonine; lysine, arginine; and tyrosine, phenylalanine.

Certain amino acids may be substituted for other amino acids in a protein structure without appreciable loss of interactive binding capacity with structures such as, for example, antigen-binding regions of antibodies or binding sites on substrate molecules or binding sites on proteins interacting with the target. Since it is the interactive capacity and nature of a protein which defines that protein's biological functional activity, certain amino acid substitutions can be made in a protein sequence and its underlying DNA coding sequence and nevertheless obtain a protein with like properties. In making such changes, the hydropathic index of amino acids may be considered. The importance of the hydrophobic amino acid index in conferring interactive biological function on a protein is generally understood in the art (Kyte and Doolittle, J. Mol. Biol. 157: 105-132, 1982). Alternatively, the substitution of like amino acids can be made effectively on the basis of hydrophilicity. The importance of hydrophilicity in conferring interactive biological function of a protein is generally understood in the art (U.S. Pat. No. 4,554,101). The use of the hydrophobic index or hydrophilicity in designing polypeptides is further discussed in U.S. Pat. No. 5,691,198.

The length of the polypeptide sequences compared for homology will generally be at least about 16 amino acids, usually at least about 20 residues, more usually at least about 24 residues, typically at least about 28 residues and preferably more than about 35 residues.

The nucleotide sequence encoding SOCS-3 is set forth in SEQ ID NOs:1 and 2, respectively for human SOCS-3 and SEQ ID NOs:3 and 4, respectively for mouse SOCS-3.

The polypeptide sequences for SOCS-3 are set forth in SEQ ID NO:2 (murine) and SEQ ID NO:4 (human). Full length polypeptides, or parts thereof, may be used in accordance with the present invention to identify agonists or antagonists of SOCS-3 expression or activity. Preferred polypeptides include the polypeptides set forth in SEQ ID NO:2 and SEQ ID NO:4, and polypeptides comprising the SH₂ domain of SOCS-3 (amino acids 45 to 127 of SEQ ID NO:2 and amino acids 45 to 127 of SEQ ID NO:4) and/or the SOCS-box motif (amino acids 186 to 221 of SEQ ID NO:2 and amino acids 186 to 221 of SEQ ID NO:4).

The nucleotide sequences corresponding to the SH₂ domain and SOCS-box motif are nucleotides 133 to 381 and 556 to 663 of SEQ ID NO:1 and 150 to 398 and 572 to 680 of SEQ ID NO:3. Preferred nucleotide sequences of the present invention include the nucleotide sequences set forth in SEQ ID NO:1 and SEQ ID NO:3 and nucleotide sequences comprising nucleotides 133 to 381 and/or 556 to 663 of SEQ ID NO:1 and 150 to 398 and/or 572 to 680 of SEQ ID NO:3.

The present invention further contemplates chemical analogs of the target molecules. Again, these are generally antagonistic or agonistic to target activity.

Analogs contemplated herein include but are not limited to modification to side chains, incorporating of unnatural amino acids and/or their derivatives during peptide, polypeptide or protein synthesis and the use of crosslinkers and other methods which impose conformational constraints on the proteinaceous molecule or their analogs.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids, contemplated herein is shown in Table 3.

TABLE 3
Codes for non-conventional amino acids

| Non-conventional amino acid | Code |
|---|---|
| α-aminobutyric acid | Abu |
| α-amino-α-methylbutyrate | Mgabu |
| aminocyclopropanecarboxylate | Cpro |
| aminoisobutyric acid | Aib |
| aminonorbornylcarboxylate | Norb |
| cyclohexylalanine | Chexa |
| cyclopentylalanine | Cpen |
| D-alanine | Dal |
| D-arginine | Darg |
| D-aspartic acid | Dasp |
| D-cysteine | Dcys |
| D-glutamine | Dgln |
| D-glutamic acid | Dglu |
| D-histidine | Dhis |
| D-isoleucine | Dile |
| D-leucine | Dleu |
| D-lysine | Dlys |
| D-methionine | Dmet |
| D-ornithine | Dorn |
| D-phenylalanine | Dphe |
| D-proline | Dpro |
| D-serine | Dser |
| D-threonine | Dthr |
| D-tryptophan | Dtrp |
| D-tyrosine | Dtyr |
| D-valine | Dval |
| D-α-methylalanine | Dmala |
| D-α-methylarginine | Dmarg |
| D-α-methylasparagine | Dmasn |
| D-α-methylaspartate | Dmasp |
| D-α-methylcysteine | Dmcys |
| D-α-methylglutamine | Dmgln |
| D-α-methylhistidine | Dmhis |
| D-α-methylisoleucine | Dmile |
| D-α-methylleucine | Dmleu |
| D-α-methyllysine | Dmlys |
| D-α-methylmethionine | Dmmet |
| D-α-methylornithine | Dmorn |
| D-α-methylphenylalanine | Dmphe |
| D-α-methylproline | Dmpro |
| D-α-methylserine | Dmser |
| D-α-methylthreonine | Dmthr |
| D-α-methyltryptophan | Dmtrp |
| D-α-methyltyrosine | Dmty |
| D-α-methylvaline | Dmval |
| D-N-methylalanine | Dnmala |
| D-N-methylarginine | Dnmarg |
| D-N-methylasparagine | Dnmasn |
| D-N-methylaspartate | Dnmasp |
| D-N-methylcysteine | Dnmcys |
| D-N-methylglutamine | Dnmgln |
| D-N-methylglutamate | Dnmglu |
| D-N-methylhistidine | Dnmhis |
| D-N-methylisoleucine | Dnmile |
| D-N-methylleucine | Dmnleu |
| D-N-methyllysine | Dnmlys |
| N-methylcyclohexylalanine | Nmchexa |
| D-N-methylornithine | Dnmorn |
| N-methylglycine | Nala |
| N-methylaminoisobutyrate | Nmaib |
| N-(1-methylpropyl)glycine | Nile |
| N-(2-methylpropyl)glycine | Nleu |
| D-N-methyltryptophan | Dnmtrp |
| D-N-methyltyrosine | Dnmtyr |
| D-N-methylvaline | Dnmval |
| γ-aminobutyric acid | Gabu |
| L-t-butylglycine | Tbug |
| L-ethylglycine | Etg |
| L-homophenylalanine | Hphe |
| L-α-methylarginine | Marg |
| L-α-methylaspartate | Masp |
| L-α-methylcysteine | Mcys |
| L-α-methylglutamine | Mgln |
| L-α-methylhistidine | Mhis |
| L-α-methylisoleucine | Mile |
| L-α-methylleucine | Mleu |
| L-α-methylmethionine | Mmet |
| L-α-methylnorvaline | Mnva |
| L-α-methylphenylalanine | Mphe |
| L-α-methylserine | Mser |
| L-α-methyltryptophan | Mtrp |
| L-α-methylvaline | Mval |
| N-(N-(2,2-diphenylethyl)carbamylmethyl)glycine | Nnbhm |
| 1-carboxy-1-(2,2-diphenylethylamino)cyclopropane | Nmbc |
| L-N-methylalanine | Nmala |
| L-N-methylarginine | Nmarg |
| L-N-methylasparagine | Nmasn |
| L-N-methylaspartic acid | Nmasp |
| L-N-methylcysteine | Nmcys |
| L-N-methylglutamine | Nmgln |
| L-N-methylglutamic acid | Nmglu |
| L-Nmethylhistidine | Nmhis |
| L-N-methylisolleucine | Nmile |
| L-N-methylleucine | Nmleu |
| L-N-methyllysine | Nmlys |
| L-N-methylmethionine | Nmmet |
| L-N-methylnorleucine | Nmnle |
| L-N-methylnorvaline | Nmnva |
| L-N-methylornithine | Nmorn |
| L-N-methylphenylalanine | Nmphe |
| L-N-methylproline | Nmpro |
| L-N-methylserine | Nmser |
| L-N-methylthreonine | Nmthr |
| L-N-methyltryptophan | Nmtrp |
| L-N-methyltyrosine | Nmtyr |
| L-N-methylvaline | Nmval |
| L-N-methylethylglycine | Nmetg |
| L-N-methyl-t-butylglycine | Nmtbug |
| L-norleucine | Nle |
| L-norvaline | Nva |
| α-methyl-aminoisobutyrate | Maib |
| α-methyl-γ-aminobutyrate | Mgabu |
| α-methylcyclohexylalanine | Mchexa |
| α-methylcylcopentylalanine | Mcpen |
| α-methyl-α-napthylalanine | Manap |
| α-methylpenicillamine | Mpen |
| N-(4-aminobutyl)glycine | Nglu |
| N-(2-aminoethyl)glycine | Naeg |
| N-(3-aminopropyl)glycine | Norn |
| N-amino-α-methylbutyrate | Nmaabu |
| α-napthylalanine | Anap |
| N-benzylglycine | Nphe |
| N-(2-carbamylethyl)glycine | Ngln |
| N-(carbamylmethyl)glycine | Nasn |
| N-(2-carboxyethyl)glycine | Nglu |
| N-(carboxymethyl)glycine | Nasp |
| N-cyclobutylglycine | Ncbut |
| N-cycloheptylglycine | Nchep |
| N-cyclohexylglycine | Nchex |
| N-cyclodecylglycine | Ncdec |
| N-cylcododecylglycine | Ncdod |
| N-cyclooctylglycine | Ncoct |
| N-cyclopropylglycine | Ncpro |
| N-cycloundecylglycine | Ncund |
| N-(2,2-diphenylethyl)glycine | Nbhm |
| N-(3,3-diphenylpropyl)glycine | Nbhe |
| N-(3-guanidinopropyl)glycine | Narg |
| N-(1-hydroxyethyl)glycine | Nthr |
| N-(hydroxyethyl))glycine | Nser |
| N-(imidazolylethyl))glycine | Nhis |
| N-(3-indolylyethyl)glycine | Nhtrp |
| N-methyl-γ-aminobutyrate | Nmgabu |
| D-N-methylmethionine | Dnmmet |
| N-methylcyclopentylalanine | Nmcpen |
| D-N-methylphenylalanine | Dnmphe |
| D-N-methylproline | Dnmpro |
| D-N-methylserine | Dnmser |
| D-N-methylthreonine | Dnmthr |
| N-(1-methylethyl)glycine | Nval |
| N-methyla-napthylalanine | Nmanap |
| N-methylpenicillamine | Nmpen |
| N-(p-hydroxyphenyl)glycine | Nhtyr |
| N-(thiomethyl)glycine | Ncys |
| penicillamine | Pen |
| L-α-methylalanine | Mala |
| L-α-methylasparagine | Masn |
| L-α-methyl-t-butylglycine | Mtbug |
| L-methylethylglycine | Metg |
| L-α-methylglutamate | Mglu |
| L-α-methylhomophenylalanine | Mhphe |
| N-(2-methylthioethyl)glycine | Nmet |
| L-α-methyllysine | Mlys |
| L-α-methylnorleucine | Mnle |
| L-α-methylornithine | Morn |
| L-α-methylproline | Mpro |
| L-α-methylthreonine | Mthr |
| L-α-methyltyrosine | Mtyr |
| L-N-methylhomophenylalanine | Nmhphe |
| N-(N-(3,3-diphenylpropyl)carbamylmethyl)glycine | Nnbhe |

Crosslinkers can be used, for example, to stabilize 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids, introduction of double bonds between C_{α} and C_{β} atoms of amino acids and the formation of cyclic peptides or analogs by introducing covalent bonds such as forming an amide bond between the N and C termini, between two side chains or between a side chain and the N or C terminus.

The terms “peptide mimetic”, “target mimetic” or “mimetic” are intended to refer to a substance which has some chemical similarity to the target but which antagonizes or agonizes or mimics the target. A peptide mimetic may be a peptide-containing molecule that mimics elements of protein secondary structure (Johnson et al., “Peptide Turn Mimetics” in Biotechnology and Pharmacy, Pezzuto et al., Eds., Chapman and Hall, New York, 1993). The underlying rationale behind the use of peptide mimetics is that the peptide backbone of proteins exists chiefly to orient amino acid side chains in such a way as to facilitate molecular interactions such as those of antibody and antigen, enzyme and substrate or scaffolding proteins. A peptide mimetic is designed to permit molecular interactions similar to the natural molecule and, hence, compete for molecules which might otherwise interact with SOCS-3.

Again, the compounds of the present invention may be selected to interact with a target alone or single or multiple compounds may be used to affect multiple targets.

The target polypeptide or fragment employed in such a test may either be free in solution, affixed to a solid support, or borne on a cell surface. One method of drug screening utilizes eukaryotic or prokaryotic host cells which are stably transformed with recombinant polynucleotides expressing the polypeptide or fragment, preferably in competitive binding assays. Such cells, either in viable or fixed form, can be used for standard binding assays. One may measure, for example, the formation of complexes between a target or fragment and the agent being tested, or examine the degree to which the formation of a complex between a target or fragment and a known ligand is aided or interfered with by the agent being tested.

A substance identified as a modulator of target function or gene activity may be a peptide or non-peptide in nature. Non-peptide “small molecules” are often preferred for many in vivo pharmaceutical uses. Accordingly, a mimetic or mimic of the substance (particularly if a peptide) may be designed for pharmaceutical use.

The designing of mimetics to a pharmaceutically active compound is a known approach to the development of pharmaceuticals based on a “lead” compound. This might be desirable where the active compound is difficult or expensive to synthesize or where it is unsuitable for a particular method of administration, e.g. peptides are unsuitable active agents for oral compositions as they tend to be quickly degraded by proteases in the alimentary canal. Mimetic design, synthesis and testing is generally used to avoid randomly screening large numbers of molecules for a target property.

There are several steps commonly taken in the design of a mimetic from a compound having a given target property. First, the particular parts of the compound that are critical and/or important in determining the target property are determined. In the case of a peptide, this can be done by systematically varying the amino acid residues in the peptide, e.g. by substituting each residue in turn. Alanine scans of peptides are commonly used to refine such peptide motifs. These parts or residues constituting the active region of the compound are known as its “pharmacophore”.

Once the pharmacophore has been found, its structure is modeled according to its physical properties, e.g. stereochemistry, bonding, size and/or charge, using data from a range of sources, e.g. spectroscopic techniques, x-ray diffraction data and NMR. Computational analysis, similarity mapping (which models the charge and/or volume of a pharmacophore, rather than the bonding between atoms) and other techniques can be used in this modeling process.

In a variant of this approach, the three-dimensional structure of the ligand and its binding partner are modeled. This can be especially useful where the ligand and/or binding partner change conformation on binding, allowing the model to take account of this in the design of the mimetic. Modeling can be used to generate inhibitors which interact with the linear sequence or a three-dimensional configuration.

A template molecule is then selected onto which chemical groups which mimic the pharmacophore can be grafted. The template molecule and the chemical groups grafted onto it can conveniently be selected so that the mimetic is easy to synthesize, is likely to be pharmacologically acceptable, and does not degrade in vivo, while retaining the biological activity of the lead compound. Alternatively, where the mimetic is peptide-based, further stability can be achieved by cyclizing the peptide, increasing its rigidity. The mimetic or mimetics found by this approach can then be screened to see whether they have the target property, or to what extent they exhibit it. Further optimization or modification can then be carried out to arrive at one or more final mimetics for in vivo or clinical testing.

The goal of rational drug design is to produce structural analogs of biologically active polypeptides of interest or of small molecules with which they interact (e.g. agonists, antagonists, inhibitors or enhancers) in order to fashion drugs which are, for example, more active or stable forms of the polypeptide, or which, e.g. enhance or interfere with the function of a polypeptide in vivo. See, e.g. Hodgson (Bio/Technology 9: 19-21, 1991). In one approach, one first determines the three-dimensional structure of a protein of interest (i.e. G-CSF or SOCS-3) by x-ray crystallography, by computer modeling or most typically, by a combination of approaches. Useful information regarding the structure of a polypeptide may also be gained by modeling based on the structure of homologous proteins. An example of rational drug design is the development of HIV protease inhibitors (Erickson et al., Science 249: 527-533, 1990). In addition, target molecules may be analyzed by an alanine scan (Wells, Methods Enzymol. 202: 2699-2705, 1991). In this technique, an amino acid residue is replaced by Ala and its effect on the peptide's activity is determined. Each of the amino acid residues of the peptide is analyzed in this manner to determine the important regions of the peptide.

It is also possible to isolate a target-specific antibody, selected by a functional assay and then to solve its crystal structure. In principle, this approach yields a pharmacore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced banks of peptides. Selected peptides would then act as the pharmacore.

Two-hybrid screening is also useful in identifying other members of a biochemical or genetic pathway associated with a target. Two-hybrid screening conveniently uses Saccharomyces cerevisiae and Saccharomyces pombe. Target interactions and screens for inhibitors can be carried out using the yeast two-hybrid system, which takes advantage of transcriptional factors that are composed of two physically separable, functional domains. The most commonly used is the yeast GAL4 transcriptional activator consisting of a DNA binding domain and a transcriptional activation domain. Two different cloning vectors are used to generate separate fusions of the GAL4 domains to genes encoding potential binding proteins. The fusion proteins are co-expressed, targeted to the nucleus and if interactions occur, activation of a reporter gene (e.g. lacZ) produces a detectable phenotype. In the present case, for example, S. cerevisiae is co-transformed with a library or vector expressing a cDNA GAL4 activation domain fusion and a vector expressing a G-CSF-GAL4 or SOCS-3-GAL4 binding domain fusion. If lacZ is used as the reporter gene, co-expression of the fusion proteins will produce a blue color. Small molecules or other candidate compounds which interact with a target will result in loss of color of the cells. This system can be used to screen for small molecules that inhibit the target function of targets such as SOCS-3. Reference may be made to the yeast two-hybrid systems as disclosed by Munder et al. (Appl. Microbiol. Biotechnol. 52(3): 311-320, 1999) and Young et al., Nat. Biotechnol. 16(10): 946-950, 1998). Molecules thus identified by this system are then re-tested in animal cells. A similar approach may also be used to locate agonists of SOCS-3.

The present invention extends to a genetic approach for up- or down-regulating SOCS-3 levels or activity. In one example, nucleic acid molecules which encode SOCS-3 or which are used to up- or down-regulate the genes encoding SOCS-3 are introduced to cells.

The terms “nucleic acids”, “nucleotide” and “polynucleotide” include RNA, cDNA, genomic DNA, synthetic forms and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog (such as the morpholine ring), internucleotide modifications such as uncharged linkages (e.g. methyl phosphonates, phosphotriesters, phosphoamidates, carbamates, etc.), charged linkages (e.g. phosphorothioates, phosphorodithioates, etc.), pendent moieties (e.g. polypeptides), intercalators (e.g. acridine, psoralen, etc.), chelators, alkylators and modified linkages (e.g. α-anomeric nucleic acids, etc.). Also included are synthetic molecules that mimic polynucleotides in their ability to bind to a designated sequence via hydrogen binding and other chemical interactions. Such molecules are known in the art and include, for example, those in which peptide linkages substitute for phosphate linkages in the backbone of the molecule.

Antisense polynucleotide sequences, for example, are useful in preventing or diminishing the expression of a genetic sequence or locus encoding SOCS-3. The nucleotide sequence encoding human SOCS-3 is set forth in SEQ ID NO:1. An example of a homolog is murine SOCS-3 which is encoded by SEQ ID NO:3. Polynucleotide vectors, for example, containing all or a portion of a target SOCS-3 locus may be placed under the control of a promoter in an antisense orientation and introduced into a cell. Expression of such an antisense construct within a cell will interfere with target transcription and/or translation.

Furthermore, co-suppression and mechanisms to induce RNAi or siRNA may also be employed. Such techniques may be useful to inhibit genes which encode or promote SOCS-3 gene expression. Alternatively, antisense or sense molecules may be directly administered. In this latter embodiment, the antisense or sense molecules may be formulated in a composition and then administered by any number of means to target cells.

A variation on antisense and sense molecules involves the use of morpholinos, which are oligonucleotides composed of morpholine nucleotide derivatives and phosphorodiamidate linkages (for example, Summerton and Weller, Antisense and Nucleic Acid Drug Development 7: 187-195, 1997). Such compounds are injected into embryos and the effect of interference with mRNA is observed.

In one embodiment, the present invention employs compounds such as oligonucleotides and similar species for use in modulating the function or effect of nucleic acid molecules encoding SOCS-3, i.e. the oligonucleotides induce transcriptional or post-transcriptional gene silencing. This is accomplished by providing oligonucleotides which specifically hybridize with one or more nucleic acid molecules encoding SOCS-3. The oligonucleotides may be provided directly to a cell or generated within the cell. As used herein, the terms “target nucleic acid” and “nucleic acid molecule encoding SOCS-3” have been used for convenience to encompass DNA encoding G-CSF or SOCS-3, RNA (including pre-mRNA and mRNA or portions thereof) transcribed from such DNA, and also cDNA derived from such RNA. The hybridization of a compound of the subject invention with its target nucleic acid is generally referred to as “antisense”. Consequently, the preferred mechanism believed to be included in the practice of some preferred embodiments of the invention is referred to herein as “antisense inhibition.” Such antisense inhibition is typically based upon hydrogen bonding-based hybridization of oligonucleotide strands or segments such that at least one strand or segment is cleaved, degraded, or otherwise rendered inoperable. In this regard, it is presently preferred to target specific nucleic acid molecules and their functions for such antisense inhibition.

The functions of DNA to be interfered with can include replication and transcription. Replication and transcription, for example, can be from an endogenous cellular template, a vector, a plasmid construct or otherwise. The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA. One preferred result of such interference with target nucleic acid function is modulation of the expression of the growth hormone gene. In the context of the present invention, “modulation” and “modulation of expression” mean either an increase (stimulation) or a decrease (inhibition) in the amount or levels of a nucleic acid molecule encoding the gene, e.g., DNA or RNA. Inhibition is often the preferred form of modulation of expression and mRNA is often a preferred target nucleic acid.

In the context of this invention, “hybridization” means the pairing of complementary strands of oligomeric compounds. In the present invention, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances.

An antisense compound is specifically hybridizable when binding of the compound to the target nucleic acid interferes with the normal function of the target nucleic acid to cause a loss of activity, and there is a sufficient degree of complementarity to avoid non-specific binding of the antisense compound to non-target nucleic acid sequences under conditions in which specific binding is desired, i.e. under physiological conditions in the case of in vivo assays or therapeutic treatment, and under conditions in which assays are performed in the case of in vitro assays.

“Complementary” as used herein, refers to the capacity for precise pairing between two nucleobases of an oligomeric compound. For example, if a nucleobase at a certain position of an oligonucleotide (an oligomeric compound), is capable of hydrogen bonding with a nucleobase at a certain position of a target nucleic acid, said target nucleic acid being a DNA, RNA, or oligonucleotide molecule, then the position of hydrogen bonding between the oligonucleotide and the target nucleic acid is considered to be a complementary position. The oligonucleotide and the further DNA, RNA, or oligonucleotide molecule are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligonucleotide and a target nucleic acid.

According to the present invention, compounds include antisense oligomeric compounds, antisense oligonucleotides, ribozymes, external guide sequence (EGS) oligonucleotides, alternate splicers, primers, probes, and other oligomeric compounds which hybridize to at least a portion of the target nucleic acid. As such, these compounds may be introduced in the form of single-stranded, double-stranded, circular or hairpin oligomeric compounds and may contain structural elements such as internal or terminal bulges or loops. Once introduced to a system, the compounds of the invention may elicit the action of one or more enzymes or structural proteins to effect modification of the target nucleic acid. One non-limiting example of such an enzyme is RNAse H, a cellular endonuclease which cleaves the RNA strand of an RNA:DNA duplex. It is known in the art that single-stranded antisense compounds which are “DNA-like” elicit RNAse H. Activation of RNase H, therefore, results in cleavage of the RNA target, thereby greatly enhancing the efficiency of oligonucleotide-mediated inhibition of gene expression. Similar roles have been postulated for other ribonucleases such as those in the RNase III and ribonuclease L family of enzymes.

While the preferred form of antisense compound is a single-stranded antisense oligonucleotide, in many species the introduction of double-stranded structures, such as double-stranded RNA (dsRNA) molecules, has been shown to induce potent and specific antisense-mediated reduction of the function of a gene or its associated gene products. This phenomenon occurs in both plants and animals.

In the context of the subject invention, the term “oligomeric compound” refers to a polymer or oligomer comprising a plurality of monomeric units. In the context of this invention, the term “oligonucleotide” refers to an oligomer or polymer of ribonucleic acid (RNA) or deoxyribonucleic acid (DNA) or mimetics, chimeras, analogs and homologs thereof. This term includes oligonucleotides composed of naturally occurring nucleobases, sugars and covalent internucleoside (backbone) linkages as well as oligonucleotides having non-naturally occurring portions which function similarly. Such modified or substituted oligonucleotides are often preferred over native forms because of desirable properties such as, for example, enhanced cellular uptake, enhanced affinity for a target nucleic acid and increased stability in the presence of nucleases.

While oligonucleotides are a preferred form of the compounds of this invention, the present invention comprehends other families of compounds as well, including but not limited to oligonucleotide analogs and mimetics such as those herein described.

The compounds in accordance with this invention preferably comprise from about 8 to about 80 nucleobases (i.e. from about 8 to about 80 linked nucleosides). One of ordinary skill in the art will appreciate that the invention embodies compounds of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases in length. Although 80 nucleobases is given as an upper range, any length from 8 to the full length gene transcript may be provided.

The open reading frame (ORF) or “coding region” which is known in the art to refer to the region between the translation initiation codon and the translation termination codon, is a region which may be targeted effectively. Within the context of the present invention, one region is the intragenic region encompassing the translation initiation or termination codon of the open reading frame (ORF) of a gene.

Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA (or corresponding nucleotides on the gene), and the 3′ untranslated region (3′UTR), known in the art to refer to the portion of an mRNA in the 3′ direction from the translation termination codon, and thus including nucleotides between the translation termination codon and 3′ end of an mRNA (or corresponding nucleotides on the gene). The 5′ cap site of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap site. It is also preferred to target the 5′ cap region.

Although some eukaryotic mRNA transcripts are directly translated, many contain one or more regions, known as “introns”, which are excised from a transcript before it is translated. The remaining (and, therefore, translated) regions are known as “exons” and are spliced together to form a continuous mRNA sequence. Targeting splice sites, i.e. intron-exon junctions or exon-intron junctions, may also be particularly useful in situations where aberrant splicing is implicated in disease, or where an overproduction of a particular splice product is implicated in disease. Aberrant fusion junctions due to rearrangements or deletions are also preferred target sites. mRNA transcripts produced via the process of splicing of two (or more) mRNAs from different gene sources are known as “fusion transcripts”. It is also known that introns can be effectively targeted using antisense compounds targeted to, for example, DNA or pre-mRNA.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are generally preferred. In addition, linear compounds may have internal nucleobase complementarity and may, therefore, fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

In an alternative embodiment, genetic constructs including DNA vaccines are used to generate antisense molecules in vivo. Furthermore, many of the preferred features described above are appropriate for sense nucleic acid molecules or for gene therapy applications to promote SOCS-3 gene expression. For example, genetic constructs may be administered which generate elevated levels of SOCS-3.

Following identification of a substance which modulates SOCS-3 activity or gene expression, it may be manufactured and/or used in a preparation, i.e. in the manufacture or formulation or a composition such as a medicament, pharmaceutical composition or drug. These may be administered to individuals in a method of treatment or prophylaxis or regenerative therapy. Alternatively, they may be incorporated into a patch or slow release capsule or implant.

Thus, the present invention extends, therefore, to a pharmaceutical composition, medicament, drug or other composition including a patch or slow release formulation comprising an agonist or antagonist of target activity or gene expression. Another aspect of the present invention contemplates a method comprising administration of such a composition to a patient such as for treatment or prophylaxis of a range of G-CSF-induced cellular responses. Up-regulation of G-CSF-induced signaling is proposed to be useful in facilitating neutrophil recovery after myelosuppressive chemotherapy, radiotherapy or chemoradiotherapy, inducing mobilization of stem and progenitor cells and in the treatment of prophylaxis of bacterial, viral or fungal infection or recovery therefrom.

Down-regulation of G-CSF such as following administration of agonists of SOCS-3 is proposed to be useful in reducing unwanted clinical sequelae of inflammatory processes characterized by neutrophil accumulation and activation. Examples of such clinical sequelae include engraftment syndrome following allogenic or autologous stem cell transplantation, pulmonary inflammation such as observed after recovery from neutropenia, acute arthritis, inflammatory bowel disease and Sweets syndrome. The compounds of the present invention may also be used in the manufacture of a medicament for the treatment or prophylaxis of a G-CSF-induced cellular response. Furthermore, the present invention contemplates a method of making a pharmaceutical composition comprising admixing a compound of the instant invention with a pharmaceutically acceptable excipient, vehicle or carrier, and optionally other ingredients. Where multiple compositions are provided, then such compositions may be given simultaneously or sequentially. Sequential administration includes administration within nanoseconds, seconds, minutes, hours or days. Preferably, within seconds or minutes.

Accordingly, another aspect of the present invention contemplates a method for the treatment or prophylaxis of a G-CSF-induced cellular response in an animal, said method comprising administering to said animal an effective amount of a compound as described herein or a composition comprising same.

Preferably, the animal is a mammal such as a human or other primate or lower primate or laboratory test animal such as a mouse, rat, rabbit, guinea pig, hamster, zebrafish or amphibian.

According to the present invention, a method is also provided of supplying wild-type or mutant target gene function to a cell. This is particularly useful when generating an animal model. Alternatively, it may be part of a gene therapy approach. A target gene or a part of the gene may be introduced into the cell in a vector such that the gene remains extrachromosomal. In such a situation, the gene will be expressed by the cell from the extrachromosomal location. If a gene portion is introduced and expressed in a cell carrying a mutant target allele, the gene portion should encode a part of the target protein. Vectors for introduction of genes both for recombination and for extrachromosomal maintenance are known in the art and any suitable vector may be used. Methods for introducing DNA into cells such as electroporation calcium phosphate co-precipitation and viral transduction are known in the art.

Gene transfer systems known in the art may be useful in the practice of genetic manipulation. These include viral and non-viral transfer methods. A number of viruses have been used as gene transfer vectors or as the basis for preparing gene transfer vectors, including papovaviruses (e.g. SV40, Madzak et al., J. Gen. Virol. 73: 1533-1536, 1992), adenovirus (Berkner, Curr. Top. Microbiol. Immunol. 158: 39-66, 1992; Berkner et al., BioTechniques 6; 616-629, 1988; Gorziglia and Kapikian, J. Virol. 66: 4407-4412, 1992; Quantin et al., Proc. Natl. Acad. Sci. USA 89: 2581-2584, 1992; Rosenfeld et al., Cell 68: 143-155, 1992; Wilkinson et al., Nucleic Acids Res. 20: 2233-2239, 1992; Stratford-Perricaudet et al., Hum. Gene Ther. 1: 241-256, 1990; Schneider et al., Nature Genetics 18: 180-183, 1998), vaccinia virus (Moss, Curr. Top. Microbiol. Immunol. 158: 25-38, 1992; Moss, Proc. Natl. Acad. Sci. USA 93: 11341-11348, 1996), adeno-associated virus (Muzyczka, Curr. Top. Microbiol. Immunol. 158: 97-129, 1992; Ohi et al., Gene 89: 279-282, 1990; Russell and Hirata, Nature Genetics 18: 323-328, 1998), herpesviruses including HSV and EBV (Margolskee, Curr. Top., Microbiol. Immunol. 158: 67-95, 1992; Johnson et al., J. Virol. 66: 2952-2965, 1992; Fink et al., Hum. Gene Ther. 3: 11-19, 1992; Breakefield and Geller, Mol. Neurobiol. 1: 339-371, 1987; Freese et al., Biochem. Pharmacol. 40: 2189-2199, 1990; Fink et al., Ann. Rev. Neurosci. 19: 265-287, 1996), lentiviruses (Naldini et al., Science 272: 263-267, 1996), Sindbis and Semliki Forest virus (Berglund et al., Biotechnology 11: 916-920, 1993) and retroviruses of avian (Bandyopadhyay and Temin, Mol. Cell. Biol. 4: 749-754, 1984; Petropoulos et al., J. Viol. 66: 3391-3397, 1992], murine [Miller, Curr. Top. Microbiol. Immunol. 158: 1-24, 1992; Miller et al., Mol. Cell. Biol. 5: 431-437, 1985; Sorge et al., Mol. Cell. Biol. 4: 1730-1737, 1984; Mann and Baltimore, J. Virol. 54: 401-407, 1985; Miller et al., J. Virol. 62: 4337-4345, 1988] and human [Shimada et al., J. Clin. Invest. 88: 1043-1047, 1991; Helseth et al., J. Virol. 64: 2416-2420, 1990; Page et al., J. Virol. 64: 5270-5276, 1990; Buchschacher and Panganiban, J. Virol. 66: 2731-2739, 1982] origin.

Non-viral gene transfer methods are known in the art such as chemical techniques including calcium phosphate co-precipitation, mechanical techniques, for example, microinjection, membrane fusion-mediated transfer via liposomes and direct DNA uptake and receptor-mediated DNA transfer. Viral-mediated gene transfer can be combined with direct in vivo gene transfer using liposome delivery, allowing one to direct the viral vectors to particular cells. Alternatively, the retroviral vector producer cell line can be injected into particular tissue. Injection of producer cells would then provide a continuous source of vector particles.

In an approach which combines biological and physical gene transfer methods, plasmid DNA of any size is combined with a polylysine-conjugated antibody specific to the adenovirus hexon protein and the resulting complex is bound to an adenovirus vector. The trimolecular complex is then used to infect cells. The adenovirus vector permits efficient binding, internalization and degradation of the endosome before the coupled DNA is damaged. For other techniques for the delivery of adenovirus based vectors, see U.S. Pat. No. 5,691,198.

Liposome/DNA complexes have been shown to be capable of mediating direct in vivo gene transfer. While in standard liposome preparations the gene transfer process is non-specific, localized in vivo uptake and expression have been reported in tumor deposits, for example, following direct in situ administration.

If the polynucleotide encodes a sense or antisense polynucleotide or a ribozyme or DNAzyme, expression will produce the sense or antisense polynucleotide or ribozyme or DNAzyme. Thus, in this context, expression does not require that a protein product be synthesized. In addition to the polynucleotide cloned into the expression vector, the vector also contains a promoter functional in eukaryotic cells. The cloned polynucleotide sequence is under control of this promoter. Suitable eukaryotic promoters include those described above. The expression vector may also include sequences, such as selectable markers and other sequences described herein.

Cells and animals which carry a mutant target allele (e.g. G-CSF or SOCS-3) or where one or both alleles are deleted can be used as model systems to study the G-CSF-induced cellular responses. Mice, rats, rabbits, guinea pigs, hamsters, zebrafish and amphibians are particularly useful as model systems. A particularly useful insertion is a loxP sequence flanking a target gene which can be excised by cre.

The present invention provides, therefore, a mutation in or flanking a genetic locus encoding a target. The mutation may be an insertion, deletion, substitution or addition to the target-coding sequence or its 5′ or 3′ untranslated region.

The animal model of the present invention is useful for screening for agents capable of ameliorating or mimicking the effects of a target. In one embodiment, the animal model produces low amounts of a target.

Another aspect of the present invention provides a genetically modified animal wherein said animal produces low amounts of a target relative to a non-genetically modified animal of the same species. Reference to “low amounts” includes zero amounts or up to about 10% lower than normalized amounts.

Yet another aspect of the present invention provides multiple (i.e. two or more) genes which are modified.

The animal models of the present invention may be in the form of the animals including fish or may be, for example, in the form of embryos for transplantation. The embryos are preferably maintained in a frozen state and may optionally be sold with instructions for use.

The genetically modified animals may also produce larger amounts of a target.

Accordingly, another aspect of the present invention is directed to a genetically modified animal over-expressing genetic sequences encoding a target.

A genetically modified animal includes a transgenic animal, or a “knock-out” or “knock-in” animal as well as a conditional deletion mutant. Furthermore, co-suppression may be used to induce post-transcriptional gene silencing. Co-suppression includes administration or induction of RNAi or administration or induction of siRNA or complexes comprising same.

The compounds, agents, medicaments, nucleic acid molecules and other target antagonists or agonists of the present invention can be formulated in pharmaceutical compositions which are prepared according to conventional pharmaceutical compounding techniques. See, for example, Remington's Pharmaceutical Sciences, 18^{th} Ed. (1990, Mack Publishing, Company, Easton, Pa., U.S.A.). The composition may contain the active agent or pharmaceutically acceptable salts of the active agent. These compositions may comprise, in addition to one of the active substances, a pharmaceutically acceptable excipient, carrier, buffer, stabilizer or other materials well known in the art. Such materials should be non-toxic and should not interfere with the efficacy of the active ingredient. The carrier may take a wide variety of forms depending on the form of preparation desired for administration, e.g. intravenous, oral, intrathecal, epineural or parenteral. Systemic administration may involve local or general systemic administration.

For oral administration, the compounds can be formulated into solid or liquid preparations such as capsules, pills, tablets, lozenges, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier. See for example, International Patent Publication No. WO 96/11698.

For parenteral administration, the compound may dissolved in a pharmaceutical carrier and administered as either a solution of a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

The active agent is preferably administered in a therapeutically effective amount. The actual amount administered and the rate and time-course of administration will depend on the nature and severity of the condition being treated. Prescription of treatment, e.g. decisions on dosage, timing, etc. is within the responsibility of general practitioners or specialists and typically takes account of the disorder to be treated, the condition of the individual patient, the site of delivery, the method of administration and other factors known to practitioners. Examples of techniques and protocols can be found in Remington's Pharmaceutical Sciences, supra.

Alternatively, targeting therapies may be used to deliver the active agent more specifically to certain types of cell, by the use of targeting systems such as antibodies or cell specific ligands or specific nucleic acid molecules. Targeting may be desirable for a variety of reasons, e.g. if the agent is unacceptably toxic or if it would otherwise require too high a dosage or if it would not otherwise be able to enter the target cells.

Instead of administering these agents directly, they could be produced in the target cell, e.g. in a viral vector such as described above or in a cell based delivery system such as described in U.S. Pat. No. 5,550,050 and International Patent Publication Nos. WO 92/19195, WO 94/25503, WO 95/01203, WO 95/05452, WO 96/02286, WO 96/02646, WO 96/40871, WO 96/40959 and WO 97/12635. The vector could be targeted to the target cells. The cell based delivery system is designed to be implanted in a patient's body at the desired target site and contains a coding sequence for the target agent. Alternatively, the agent could be administered in a precursor form for conversion to the active form by an activating agent produced in, or targeted to, the cells to be treated. See, for example, European Patent Application No. 0 425 731A and International Patent Publication No. WO 90/07936.

The present invention is further described by the following non-limiting Examples.

### EXAMPLE 1

### Generation of Mice with SOCS-3-Deficient Hemopoiesis

Generation of mice bearing null (SOCS-3⁻) and LoxP-flanked conditional (SOCS-3^{fl}) alleles of SOCS-3 have been previously described (Roberts et al., Proc. Natl. Acad. Sci. USA 98(16): 9324-9329, 2001; Croker et al., Nat Immunol. 4(6): 540-545, 2003) In order to generate mice with SOCS-3-deficient blood cells, transgenic mice were created in which Cre recombinase expression was restricted to cells of the hemopoietic and endothelial lineages (VavCre⁺ mice).

A 11.2 kbp plasmid, containing elements of the Vav promoter and a human CD4 reporter was digested with Sfi I and Not I excising the human CD4 reporter which was then replaced with a nls-Cre (nuclear localization signal-Cre) recombinase cassette (Ogilvy et al., Blood 94(6): 1855-1863, 1999). The pIC19H (prokaryotic) sequences were removed by restriction digestion with Hind III and the remaining 8.2 kbp fragment was purified from low-melt agarose using agarase (New England Biolabs, USA). The purified DNA was dialyzed for 12 hours in microinjection buffer (10 mM Tris/HCl pH 7.4, 0.1 mM EDTA) and adjusted to 2 μg/mL for microinjection. Eight founders were obtained from 93 potential founders following pronuclear microinjection. Based on the expression of the Vav-Cre transgene in GtROSA26 lacZ reporter mice (Soriano et al., Nat. Genet. 21(1): 70-71, 1999), three independent Vav-Cre transgenic lines (15, 48 and 71) were selected to use for intercrossing with mice bearing mutant SOCS-3 alleles.

### EXAMPLE 2

### Clonogenic Assays, FACS Analyses and Tritiated Thymidine Incorporation Assays

These were performed exactly as previously described (Alexander et al., Blood 87(6): 2162-2170, 1996; Croker et al., Immunol. Cell Biol. 80(3): 231-240, 2002).

### EXAMPLE 3

### In Vivo Responses to G-CSF

Mice were injected intraperitoneally twice daily with 2.5 μg rhG-CSF (lenograstim). These experiments were performed exactly as previously described except that mice were only injected for four days and analyzed on the fifth day (Roberts et al., Exp. Hematol. 22(12): 1156-1163, 1994).

### EXAMPLE 4

### The VavCre Transgene Targets Cre Recombinase Activity to Hemopoietic and Endothelial Cells

To determine the cell-type and tissue-distribution pattern of SOCS-3 deletion to be expected in subsequent experiments, VavCre⁺ mice were intercrossed with Gt-ROSA26lacZ reporter mice and β-galactosidase activity was used as an indicator of Cre-mediated deletion. For each of three lines, high level β-galactosidase expression was observed in all hemopoietic cell lineages and all endothelial cells. β-galactosidase activity was not observed in other adult cell types and tissues including hepatocytes, myocytes, adipocytes, fibroblasts, epithelial cells and renal parenchymal cells. Importantly, β-galactosidase expression was observed in >99% of neutrophils, macrophages, T cells and in greater than 90% of B cells and nucleated erythroid cells.

### EXAMPLE 5

### Hemopoietic Cells from VavCre⁺ SOCS-3^{-/fl} Mice are SOCS-3-Deficient

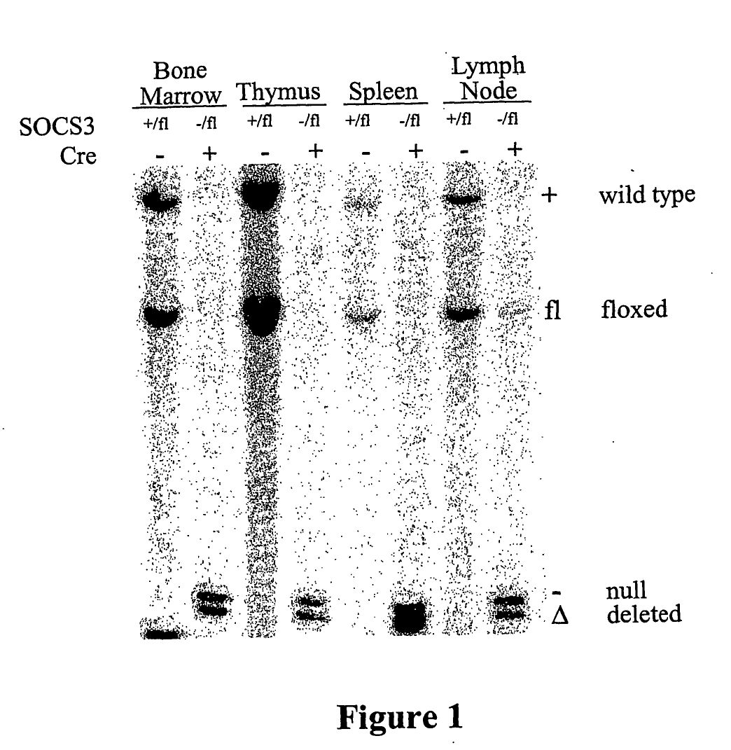

VavCre⁺ SOCS-3^{-/fl} offspring of matings between VavCre⁺ SOCS-3^{+/-} and SOCS-3^{fl/fl} mice are born in the expected Mendelian proportions, develop normally and are fertile. Genotyping of mature cells from each hemopoietic lineage demonstrated that the SOCS-3^{fl} allele had been deleted in >95% of cells (FIG. 1). VavCre⁺ SOCS-3^{-/Δ} hemopoietic cells do not have a functional SOCS-3 allele and are SOCS-3-deficient.

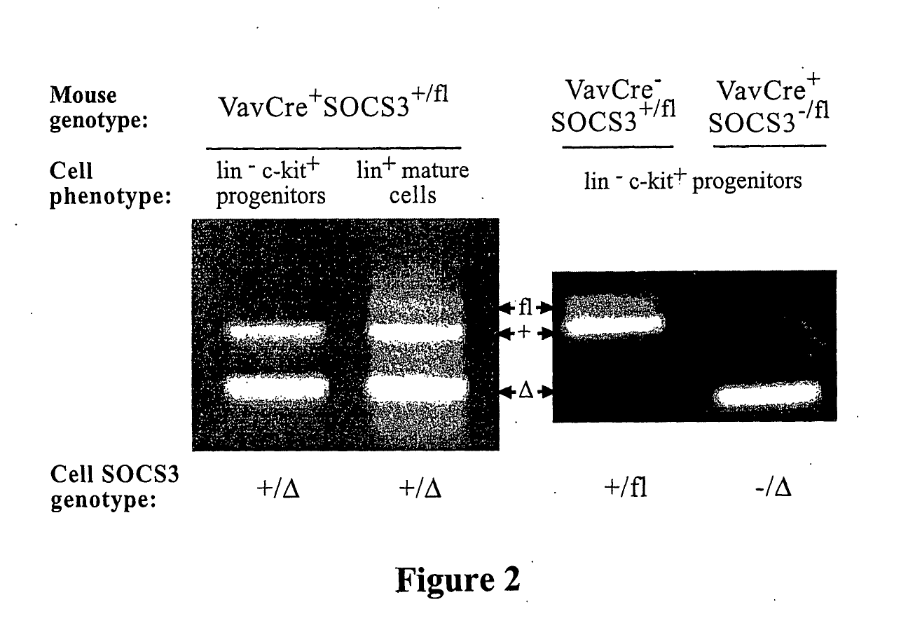

Further, hemopoietic progenitor cells are also uniformly SOCS-3-null as demonstrated by the PCR genotyping shown in FIG. 2.

### EXAMPLE 6

### Steady-State Hemopoiesis is Normal in VavCre⁺ SOCS-3^{-/fl} Mice

The great majority of 6-8 week old adult VavCre⁺ SOCS-3^{-/fl} mice display normal peripheral blood counts, bone marrow cellularity and thymic and splenic architecture and cellularity. As outlined in Tables 4 and 5, cellular content of each of these hemopoietic organs is phenotypically normal. A small number (<5%) of VavCre⁺ SOCS-3^{-/fl} mice display a neutrophil leukocytosis and splenomegaly.

TABLE 4
Peripheral blood parameters are normal in VavCre⁺ SOCS-3^{-/fl} mice with SOCS-3 deficient hemopoiesis

| | | | | | Differential | Differential | Differential | Differential |
|---|---|---|---|---|---|---|---|---|
| | | | Platelet | WCC | (×10⁹/L) | (×10⁹/L) | (×10⁹/L) | (×10⁹/L) |
| Organ | Genotype | Hct | (×10⁹/L) | (×10⁹/L) | PMN/MM | Lymph | Mono | Eosin |
| PB | Control | 49 ± 1.7 | 1342 ± 145 | 6.9 ± 1.9 | 0.6 ± 0.2 | 6.6 ± 1.5 | 0.1 ± 0.3 | 0 ± 01 |
| | VavCre⁺ SOCS-3^{-/fl} | 46 ± 2.3 | 1122 ± 306 | 4.4 ± 1.3 | 0.7 ± 0 3 | 3.6 ± 1 | 0.1 ± 0 1 | 0.1 ± 0.1 |

TABLE 5
Normal cellularity of hemopoietic organs in VavCre⁺SOCS-3^{-/fl} mice with SOCS-3 deficient hemopoiesis

| | | Cellularity | Weight | | | | | | | |
|---|---|---|---|---|---|---|---|---|---|---|
| Organ | Genotype | (×10⁶) | (mg) | Blast | Pro/Myel | PMN/MM | Lymph | Mono | Eosin | Nuc RBC |
| | Control | | | | | 9 ± 3 | 89 ± 5 | 3 ± 3 | 0 ± 1 | |
| PB | VavCre⁺ SOCS-3^{-/fl} | 4.4 ± 1.9 | | | | 15 ± 3 | 83 ± 4 | 2 ± 2 | 0 ± 1 | |
| BM | Control | 44.3 ± 14 | | 2 ± 1 | 9 ± 3 | 23 ± 5 | 34 ± 6 | 34 ± 6 | 2 ± 1 | 24 ± 2 |
| | VavCre⁺ SOCS-3^{-/fl} | 37.8 ± 4 | | 2 ± 2 | 12 ± 2 | 29 ± 13 | 25 ± 6 | 25 ± 9 | 2 ± 2 | 23 ± 1 |
| Spleen | Control | 110 ± 18 | 91 ± 13 | 1 ± 1 | 1 ± 1 | 3 ± 2 | 81 ± 6 | 81 ± 6 | | 13 ± 3 |
| | VavCre⁺ SOCS-3^{-/fl} | 137 ± 61 | 96 ± 34 | 2 ± 1 | 3 ± 1 | 4 ± 1 | 63 ± 15 | 63 ± 15 | | 25 ± 13 |

Results represent Mean ± SD or results from 4-6 mice of each genotype. Control mice include VavCre⁻SOCS-3^{+/fl} mice and VavCre⁻SOCS-3^{+/fl} mice.

### EXAMPLE 7

### Enhanced G-CSF-Induced Colony-Formation by SOCS-3-Deficient Bone Marrow Cells

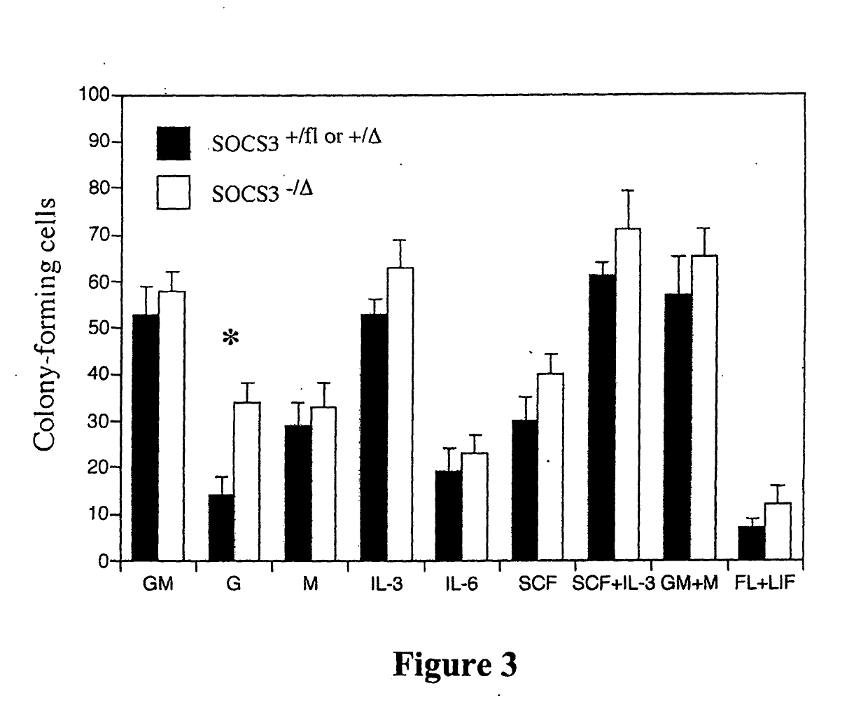

The total number of myeloid progenitor cells arising from the bone marrow population when maximally stimulated with the combination of SCF plus IL-3 is normal in VavCre⁺ SOCS-3^{-/fl} mice. However, a selective increase in the number of clonogenic cells capable of forming colonies in response to G-CSF as a single stimulus was observed. This two-fold increase was specific for G-CSF (FIG. 3), and was observed at both supramaximal and submaximal concentrations of G-CSF.

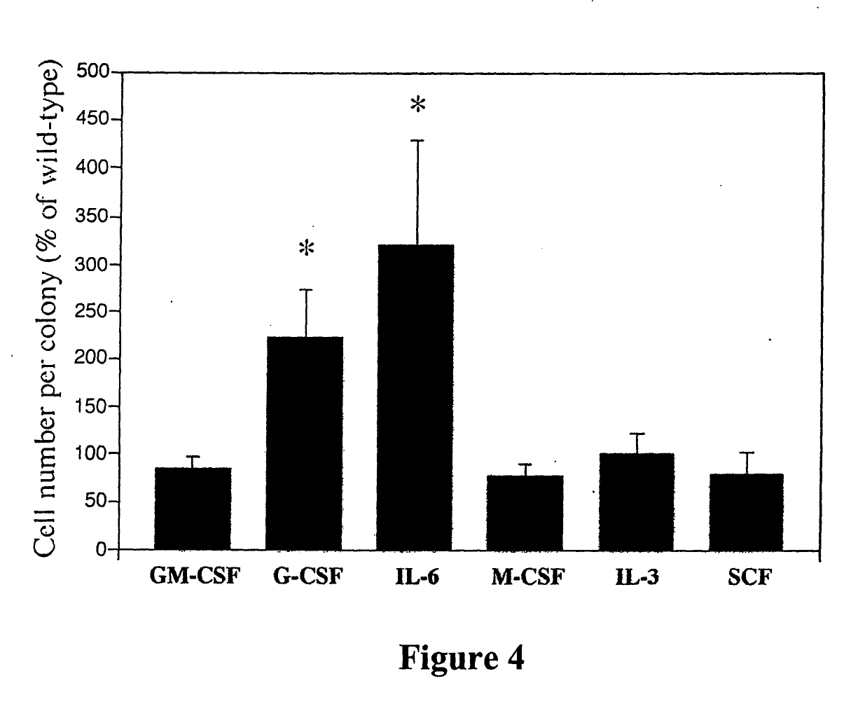

Further, the size of the emergent colonies induced by G-CSF from VavCre⁺ SOCS-3^{-/Δ} bone marrow cells was larger than that of colonies grown from control bone marrow cells (FIG. 4). Again this was a selective rather than generalized consequence of the loss of SOCS-3, as colony size was normal when cultures were stimulated with most other single cytokines or when combinations were used. However, it was not unique for G-CSF because similarly enhanced colonies were also seen with IL-6.

To investigate whether late onset neutrophilia could be a consequence of the aberrant actions of cytokines on hematopoietic progenitors, we tested the responses of hematopoietic progenitor cells in vitro to a range of hematopoietic growth factors. When maximally stimulated by GM-CSF, M-CSF, IL-3, IL-6, SCF or combinations thereof, the frequency of myeloid progenitor cells arising from the bone marrow of young, healthy mice was normal. However, a selective increase was observed in the number of vavCre⁺SOCS3^{-/Δ} clonogenic cells capable of forming colonies in response to G-CSF (FIG. 3). Strikingly, the cellular content of the colonies induced by G-CSF were 2-4 fold greater than for wild-type controls. Again, selectivity was observed for this phenotype, with no changes in the capacity for mature cell generation occurring when the progenitor cells from the same bone marrow sample were stimulated with GM-CSF, M-CSF, SCF, or combinations of these other cytokines. The increased size of the developing colonies was not unique to G-CSF stimulation, however, and was also observed following activation by IL-6.

To reduce the possibility that this two-fold increase in number of G-CSF-responsive progenitors simply reflected enrichment for committed neutrophil progenitors within the bone marrow, cultures of purified lin⁻kit⁺ progenitor cells were analysed and an increased number of G-CSF-responsive cells, both colony-forming cells and total cells, was again observed. This increased response was observed at both supramaximal and submaximal concentrations of G-CSF.

As well as apparent increased proliferation by SOCS3-deficient progenitor cells stimulated by G-CSF, a subtle shift in differentiation was also evident. There was a significant increase in the number and proportion of macrophages within these colonies, as compared with the nearly exclusively neutrophil composition of colonies derived from wild-type progenitors, with G-CSF-responsive SOCS3-deficient progenitor cells generating macrophage and granulocyte-macrophage colonies in greater proportions than wild-type progenitor cells.

### EXAMPLE 8

### SOCS-3-Deficient Myeloid Cells are Hyper-Responsive to G-CSF

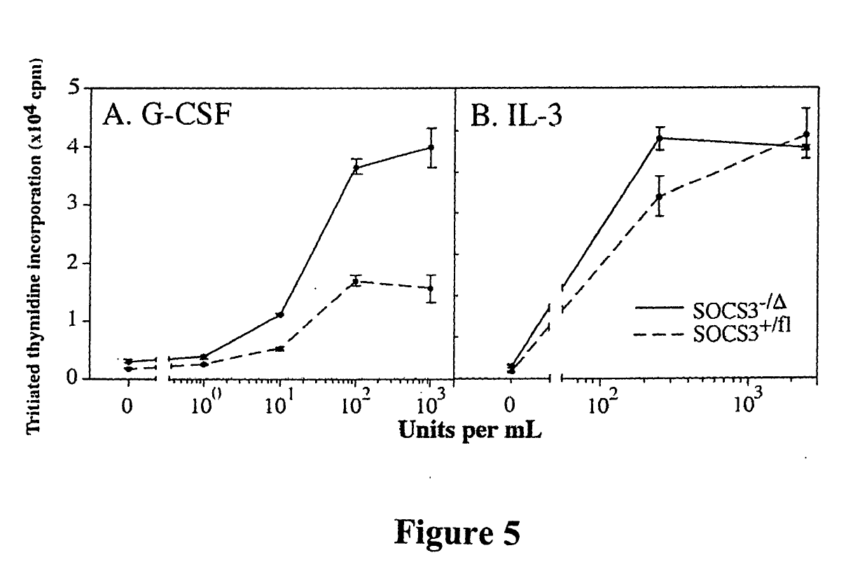

To exclude the possibility that the above observations trivially reflected aberrant composition of the myeloid progenitor cell pool in VavCre⁺ SOCS-3^{-/fl} mice, further evidence of enhanced in vitro responses to G-CSF were sought. As stated above, the distribution of morphologically-identifiable myeloid precursors is normal in VavCre⁺ SOCS-3^{-/fl} mice. Therefore, Gr-1-expressing myeloid cells were collected by fluorescence-activated cell sorting, and their proliferative responses to G-CSF were assayed by tritiated thymidine incorporation. Microscopic analyses of sorted populations confirmed there was no skewing of the composition of the precursor populations between genotypes. As illustrated in FIG. 5, thymidine incorporation by VavCre⁺ SOCS-3^{-/Δ} Gr-1⁺ cells was significantly increased at all concentrations studied. Proliferation induced by the control stimulus, IL-3, was normal, excluding an inherent proliferative advantage of cells unrelated to a specific stimulus.

Furthermore, the survival of vavCre⁺SOCS3^{-/Δ} Gr-1^{hi} cells (a population highly enriched for neutrophils and metamyelocytes, but not precursors with mitotic potential) was enhanced after 48 hours in culture in response to G-CSF.

To examine G-CSFR expression, bone marrow cells from vavCre⁺SOCS3^{-/fl} and control mice were incubated with radiolabeled G-CSF in the presence and absence of excess unlabeled G-CSF. No differences in the numbers of G-CSF were observed between genotypes (vavCre⁺SOCS3^{-/fl}, 1699±72 cpm; vavCre⁻SOCS3^{+/fl}, 1884±248 cpm), eliminating this as an explanation for the differences in cellular responses. In contrast, the duration and intensity of STAT3 phosphorylation was increased in lysates of bone marrow cells after a 15 min pulse with G-CSF in vitro. These in vitro data prove that expression of SOCS3 regulates both STAT3 phosphorylation and the proliferation and survival of myeloid cells in response to G-CSF.

### EXAMPLE 9

### SOCS-3 is Required to Negatively Regulate Emergency Granulopoiesis

The above in vitro data predict that SOCS-3 is required to negatively regulate granulopoiesis under stress conditions characterized by high levels of circulating G-CSF, i.e. emergency granulopoiesis. To mimic this situation, VavCre⁺ SOCS-3^{-/fl} mice and VavCre⁻ SOCS-3^{+/fl} controls were injected for four days with either pharmacological doses of G-CSF, or vehicle. Originally, five days of injection were planned, however G-CSF-injected VavCre⁺ SOCS-3^{-/fl} mice demonstrated severe lethargy and intermittent hind-leg paresis after four days and the experiment was modified accordingly. Wild-type mice never display such toxicity from G-CSF, even at substantially higher doses.

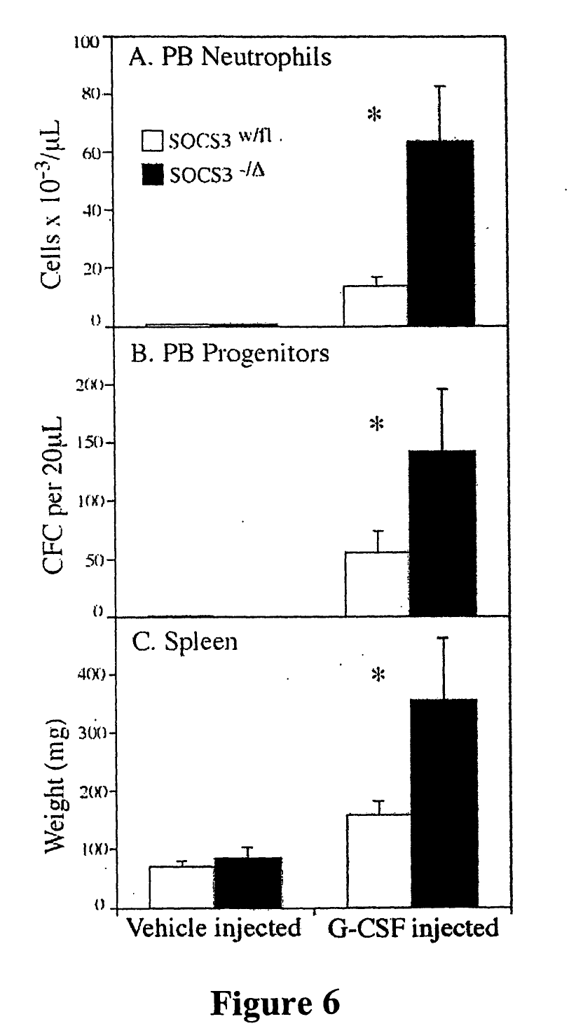

No differences were observed between genotypes when injected with vehicle only. However, in vivo responses to G-CSF were markedly accentuated in VavCre⁺ SOCS-3^{-/fl} mice. These are summarized in FIG. 6.

As well as striking neutrophilia, progenitor cell mobilisation and splenomegaly, all VavCre⁺ SOCS-3^{-/fl} mice displayed increased tissue infiltration with neutrophils on histological analysis (tissues included the liver, spinal cord and muscle). This was particularly marked in two of four mice which displayed pathological microabscess formation within the liver parenchyma. In one mouse, whole areas of bone marrow were replaced with degenerate neutrophils and their debris, reflecting inappropriate cell death. Further, evidence of pathological neutrophil death was also seen in this particular mouse in the aforementioned hepatic microabscesses.

No such changes were observed in control mice injected with G-CSF. In addition, the hematological and histological parameters of vehicle-injected vavCre⁺SOCS3^{-/fl} mice were identical to vehicle-injected control mice.

Based on our detailed survey of vavCre⁺R26R lacZ reporter mice, endothelial cells in vavCre⁺SOCS3^{-/fl} mice are very likely to be SOCS3-deficient, and therefore it is possible that the pathological responses observed in the above experiment were contributed to by loss of SOCS3 expression in tissues other than the hematopoietic system. A further caveat in interpreting these data is the hemizygosity of SOCS3 in all other tissues and the possibility that this may have contributed to the breadth of pathology observed. To confirm that the enhanced responses were principally intrinsic to the hematopoietic system, radiation chimeras were created in which the hematopoietic compartment was SOCS3-deficient and all other tissues were wild-type. SOCS3 wild-type C57BL/6.5JL (Ptprc^{a} Pep3^{b} (Ly5.1)) recipient mice were reconstituted with C57BL/6 (Ptprc^{b} Pep3^{a} (Ly5.2)) SOCS3-deficient or control fetal liver cells and then treated with the same dose of G-CSF for 4 days and analysed on day 5. Recipients of either SOCS3-deficient or control vavCre⁻ SOCS3^{+/fl} cells demonstrated efficient reconstitution by donor cells (>90% of cells in all hematopoietic lineages as judged by expression of Ly5.2).

Mice reconstituted with SOCS3-deficient hematopoietic cells were clearly hyperresponsive to G-CSF, with augmented progenitor cell mobilization and greater splenomegaly than recipients of control cells, but some differences in haematological parameters and in the degree of tissue pathology were evident between these mice and vavCre⁺SOCS3^{-/fl} mice. After G-CSF injection for 4 days, there was a near absence of mature neutrophils in the bone marrow of mice reconstituted with SOCS3-deficient cells. Surprisingly, these mature neutrophils were not detected in increased numbers in the blood as was observed for vavCre⁺SOCS3^{-/fl} mice injected with G-CSF, but rather were found in large numbers in the tissues, particularly the liver and lungs. Mice injected with G-CSF for 8 days did display a neutrophilia greater than observed for controls, ultimately recapitulating the pattern of differences observed in vavCre⁺SOCS3^{-/fl} mice. The degree of enhancement of G-CSF response observed in transplant recipients of SOCS3-deficient cells was not as marked as observed for vavCre⁺SOCS3^{-/fl} over control mice. Further, neutrophil infiltration of the spinal cord following G-CSF injection was not observed in mice reconstituted with SOCS3-deficient cells. These data indicate that while the hematopoietic cell hyperresponsiveness to G-CSF is primarily responsible for the aberrant tissue infiltration and pathology, loss of SOCS3 expression by other cells, for example endothelial cells, must also contribute to the severity of the phenotype.

### EXAMPLE 10

### Recruitment and Activation of SOCS3-Deficient Neutrophils

The pathological tissue infiltration and damage by neutrophils observed in both G-CSF injected mice with SOCS3-deficient hematopoiesis, and aging vavCre⁺SOCS3^{-/fl} mice, suggests that abnormalities in neutrophil recruitment and activity may exist. In order to analyse the survival and function of mature neutrophils in response to an inflammatory stimulus, mice were injected intraperitoneally with casein. Recruitment of neutrophils to the peritoneal cavity 3 hours following the installation of casein was normal (vavCre⁺SOCS3^{-/fl}, 5±4×10⁶ neutrophils; vavCre⁻SOCS3^{+/fl}, 5±4×10⁶ neutrophils, n=5-10 per group). The percentages of dying cells within the freshly harvested peritoneal lavages were similar between genotypes. To further investigate survival of SOCS3-deficient neutrophils after exposure to inflammatory milieux, the peritoneal exudate cells were cultured at 37° C. for 8 hours in media alone, or media supplemented with either G-CSF or GM-CSF. The survival of the inflammatory cells in media was unaffected by the absence of SOCS3 (vavCre⁺SOCS3^{-/fl}, 40±11% PI⁺; vavCre⁻SOCS3^{+/fl}, 39±10% PI⁺, n=4-7 per group), as was survival in the presence of G-CSF or GM-CSF. Finally, as a measure of the functional activity of tissue neutrophils from sites of inflammation, superoxide production was measured. Superoxide production by SOCS3-deficient neutrophils was normal in response to fMLP, with or without G-CSF priming, as well as with a maximal stimulation by PMA. No superoxide production was detected with G-CSF priming alone.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Alexander et al., Blood 87(6): 2162-2170, 1996
Alexander et al., Cell 98(5): 597-608, 1999
Avalos, Blood 88(3): 761-777, 1996
Bandyopadhyay and Temin, Mol. Cell. Biol. 4: 749-754, 1984
Berglund et al., Biotechnology 11: 916-920, 1993
Berkner et al., BioTechniques 6; 616-629, 1988
Berkner, Curr. Top. Microbiol. Immunol. 158: 39-66, 1992
Breakefield and Geller, Mol. Neurobiol. 1: 339-371, 1987
Buchschacher and Panganiban, J. Virol. 66: 2731-2739, 1982
Cai et al., Development 127: 3021-3030, 2000
Croker et al., Nat. Immunol. 4(6): 54-545, 2003
Croker et al., Immunol. Cell Biol. 80(3): 231-240, 2002
de Koning et al., Blood 87(4): 1335-1342, 1996
Erickson et al., Science 249: 527-533, 1990
Fink et al., Ann. Rev. Neurosci. 19: 265-287, 1996
Fink et al., Hum. Gene Ther. 3: 11-19, 1992
Freese et al., Biochem. Pharmacol. 40: 2189-2199, 1990
Gorziglia and Kapikian, J. Virol. 66: 4407-4412, 1992
Greenhalgh and Hilton, J. Leukoc. Biol. 70(3): 348-356, 2001
Greenhalgh et al., Molecular Endocrinology 16(6): 1394-1406, 2002
Helseth et al., J. Virol. 64: 2416-2420, 1990
Hodgson, Bio/Technology 9: 19-21, 1991
Hortner et al., J. Immunol. 169(3): 1219-1927, 2002
Johnson et al., J. Virol. 66: 2952-2965, 1992
Krebs and Hilton, J. Cell Sci. 113(16): 2813-2819, 2000
Kyte and Doolittle, J. Mol. Biol. 157: 105-132, 1982
Lee et al., Science 268: 836-844, 1995
Lindeman et al., Genes Dev. 15(13): 1631-1636, 2001
Liu et al., Genes Dev. 11(2): 179-186, 1997
Ma et al., Cell 87: 43-52, 1996
Madzak et al., J. Gen. Virol. 73: 1533-1536, 1992
Mann and Baltimore, J. Virol. 54: 401-407, 1985
Margolskee, Curr. Top., Microbiol. Immunol. 158: 67-95, 1992
Marine et al., Cell 98(5): 609-616, 1999
Marine et al., Cell 98(5): 617-627, 1999
Mathews et al., Endocrinology 123(6): 2827-2833, 1988
Matsumoto et al., Mol. Cell Biol. 19(9): 6396-6407, 1999
Metcalf et al., Nature 405(6790): 1069-1073, 2000
Miller et al., J. Virol. 62: 4337-4345, 1988
Miller et al., Mol. Cell. Biol. 5: 431-437, 1985
Miller, Curr. Top. Microbiol. Immunol. 158: 1-24, 1992
Moss, Curr. Top. Microbiol. Immunol. 158: 25-38, 1992
Moss, Proc. Natl. Acad. Sci. USA 93: 11341-11348, 1996
Munder et al., Appl. Microbiol. Biotechnol. 52(3): 311-320, 1999
Muzyczka, Curr. Top. Microbiol. Immunol. 158: 97-129, 1992
Naldini et al., Science 272: 263-267, 1996
Nicholson et al., Proc. Natl. Acad. Sci. USA 91(8): 2985-2988, 1994
Nieto et al., Neuron 29: 401-413, 2001
Ogilvy et al., Blood 94(6): 1855-1863, 1999
Ohi et al., Gene 89: 279-282, 1990
Page et al., J. Virol. 64: 5270-5276, 1990
Palmiter et al., Science 222(4625): 809-814, 1983
Petropoulos et al., J. Viol. 66: 3391-3397, 1992
Polizzotto et al., J Comp Neurol 423: 348-358, 2000
Quantin et al., Proc. Natl. Acad. Sci. USA 89: 2581-2584, 1992
Remington's Pharmaceutical Sciences, 18^{th} Ed. (1990, Mack Publishing, Company, Easton, Pa., U.S.A
Roberts et al., Exp. Hematol. 22(12): 1156-1163, 1994
Roberts et al., Proc. Natl. Acad. Sci. USA 98(16): 9324-9329, 2001
Rosenfeld et al., Cell 68: 143-155, 1992
Russell and Hirata, Nature Genetics 18: 323-328, 1998
Schneider et al., Nature Genetics 18: 180-183, 1998
Shimada et al., J. Clin. Invest. 88: 1043-1047, 1991
Sorge et al., Mol. Cell. Biol. 4: 1730-1737, 1984
Soriano et al., Nat. Genet. 21(1): 70-71, 1999
Starr et al., Nature 387(6636): 917-921, 1997
Stratford-Perricaudet et al., Hum. Gene Ther. 1: 241-256, 1990
Summerton and Weller, Antisense and Nucleic Acid Drug Development 7: 187-195, 1997
Sun et al., Cell 104: 365-376, 2001
Teglund et al., Cell 93(5): 841-850, 1998
Tian et al., Blood 84(6): 1760-1764, 1994
Tian et al., Blood 88(12): 4435-4444, 1996
Udy et al., Proc. Natl. Acad. Sci. USA 94(14): 7239-7244, 1997
Ward et al., Blood 93(1): 113-124, 1999
Wells, Methods Enzymol. 202: 2699-2705, 1991
Wilkinson et al., Nucleic Acids Res. 20: 2233-2239, 1992
Yasukawa et al., Annu. Rev. Immunol. 18: 143-164, 2000
Young et al., Nat. Biotechnol. 16(10): 946-950, 1998

1. An isolated compound which modulates G-CSF-induced cellular responses wherein the compound inhibits or activates G-CSF activity or levels via a SOCS molecule.

2. The compound of claim 1 wherein the compound up-regulates the activity or levels of SOCS-3.

3. The compound of claim 1 wherein the compound down-regulates the activity or levels of SOCS-3.

4. The compound of claim 1 wherein the compound is a nucleic acid molecule.

5. The compound of claim 4 wherein the nucleic acid molecule is an oligonucleotide.

6. The compound of claim 4 wherein the nucleic acid molecule is mRNA, RNAi, siRNA or DNA or a complex containing same.

7. The compound of claim 6 wherein the nucleic acid molecule is a sense or antisense oligonucleotide.

8. The compound of claim 1 wherein the compound is a proteinaceous molecule.

9. The compound of claim 1 wherein the compound is a non-protein chemical.

10. A pharmaceutical composition comprising an isolated compound which modulates G-CSF-induced cellular responses wherein the compound inhibits or activates G-CSF activity or levels via a SOCS molecule and one or more pharmaceutically acceptable carriers and/or diluents.

11. The pharmaceutical composition of claim 10 wherein the composition is a nucleic acid molecule.

12. The pharmaceutical composition of claim 11 wherein the composition is a gene therapy composition.

13. A method for modulating G-CSF-induced cellular responses in a mammal, said method comprising administering to said mammal an effective amount of an isolated compound which modulates G-CSF-induced cellular responses wherein the compound inhibits or activates G-CSF activity or levels via a SOCS molecule or a pharmaceutical composition comprising said isolated compound and one or more pharmaceutically acceptable carriers and/or diluents.

14. The method of claim 13 wherein the mammal is a human.

15. The method of claim 13 wherein the compound inhibits or elevates levels of SOCS-3.

16. An animal model useful for screening for compounds which modulate G-CSF-induced cellular responses, said model comprising a genetically modified animal which comprises cells which produce elevated or reduced levels of SOCS-3.

17. The animal model of claim 16 wherein the animal is a non-human primate, lower primate, mouse, rat, rabbit, sheep, goat or pig.
